(19) United States
(12) Patent Application Publication
(10) Pub. No.: US 2023/0207056 A1
(43) Pub. Date: Jun. 29, 2023

(54) AUTOMATICALLY SWITCHING VARIANT ANALYSIS MODEL VERSIONS FOR GENOMIC ANALYSIS APPLICATIONS

(71) Applicant: Illumina Software, Inc., San Diego, CA (US)

(72) Inventors: Shannon Whitmore, San Diego, CA (US); Michael Smoot, San Diego, CA (US)

(21) Appl. No.: 17/935,497

(22) Filed: Sep. 26, 2022

Related U.S. Application Data
(60) Provisional application No. 63/294,693, filed on Dec. 29, 2021.

(51) Int. Cl. G16B 30/00; G16B 40/00; G16B 50/00

(52) U.S. Cl. CPC G16B 30/00; G16B 40/00; G16B 50/00

(57) ABSTRACT

This disclosure describes methods, non-transitory computer readable media, and systems that can flexibly and efficiently change versions of a variant analysis model for different genomic analysis applications. For example, the disclosed systems can determine a particular version of a variant analysis model indicated by a genomic analysis application and can update a genomic analysis device (e.g., FPGA, CPU) by installing the indicated version of the variant analysis model. The disclosed systems can further execute a genomic analysis application to analyze nucleotide base calls utilizing the version of variant analysis model indicated by the genomic analysis application.

### CROSS-REFERENCE TO RELATED APPLICATIONS

The present application claims the benefit of, and priority to, U.S. Provisional Application No. 63/294,693, entitled “AUTOMATICALLY SWITCHING VARIANT ANALYSIS MODEL VERSIONS FOR GENOMIC ANALYSIS APPLICATIONS,” filed on Dec. 29, 2021. The aforementioned application is hereby incorporated by reference in its entirety.

### BACKGROUND

In recent years, biotechnology firms and computer science institutions have improved hardware and software for generating diagnostics and performing other genomic analyses for nucleotide sequences of genomic samples. Some existing nucleotide base sequencing platforms and/or sequencing data analysis software (together and hereinafter, existing sequencing systems) generate nucleotide base calls from nucleotide reads of a sample nucleotide sequence and/or perform a genomic analysis on nucleotide base calls for a variety of purposes. For example, an existing sequencing system can execute a sequencing analysis application for diagnostics (or for some other purpose) to screen a nucleotide sequence for a particular genetic condition by detecting specific genetic markers within nucleotide base calls (e.g., variant calls) of the sample sequence.

Despite recent advances, existing sequencing systems continue to exhibit a number of drawbacks or disadvantages. For example, many existing sequencing systems rigidly execute specific versions of genomic analysis models and cannot change versions of genomic analysis models without rendering other software applications incompatible with a different version of a model. To elaborate, existing systems often utilize genomic analysis models in the form of probabilistic models or some type of machine learning model (e.g., neural network) to perform genomic analysis on nucleotide sequences. In many cases, the genomic analysis models of these existing systems have rigid architectures that are not compatible with certain genomic analysis applications (e.g., for implementing different analysis applications and/or performing different diagnostics) without modifying or retraining the models. Such modifications or retraining can be computationally expensive and time consuming. Consequently, some existing systems experience compatibility issues and cannot execute certain sequencing, secondary, and/or tertiary analysis applications that require specific model architectures and functions to perform.

Due at least in part to their inflexible nature, some existing sequencing systems further exhibit inefficiencies and slow performance. For example, existing systems often require updating or rewriting genomic analysis applications to achieve compatibility with available hardware and software. When an existing sequencing system can execute multiple different genomic analysis applications in conjunction with a genomic analysis model, the task of updating or rewriting a genomic analysis application to become compatible with the genomic analysis model often results in sluggish execution—requiring days, weeks, or more of delay—and sometimes renders a genomic analysis application inoperable because a different version of the genomic analysis application cannot be installed. For instance, some existing sequencing systems require a client device to input computer code command lines to change or update a version of a genomic analysis model, which can often be done only with root access. Indeed, rather than quickly adapting to available architectures on the fly, existing systems sometimes require time intensive processes of retraining model architectures and/or reprogramming all or part of an application before analysis on nucleotide base calls can even begin.

### SUMMARY

This disclosure describes embodiments of methods, non-transitory computer readable media, and systems that can flexibly and efficiently switch versions of a variant analysis model for specific requirements of genomic analysis applications. For example, the disclosed systems can determine a particular version of a variant analysis model indicated by a genomic analysis application and can update a genomic analysis device by installing the indicated version of the variant analysis model. As described below, the genomic analysis device may take the form of a field programmable gate array (FPGA) or other device that operates in conjunction with a variant analysis model to perform secondary and/or tertiary analysis of nucleotide base calls. In some cases, the disclosed systems utilize a container orchestration engine to implement genomic analysis applications and to automatically (e.g., without user input specific to installation) install requisite versions of a variant analysis model. The disclosed systems can further execute a genomic analysis application to analyze nucleotide base calls (e.g., for a particular diagnostic or some other analysis) utilizing the version of variant analysis model indicated by the genomic analysis application and installed on the genomic analysis device.

### BRIEF DESCRIPTION OF THE DRAWINGS

The detailed description refers to the drawings briefly described below.

FIG. 1 illustrates a block diagram of a system environment including a model switching system in accordance with one or more embodiments.

FIG. 2 illustrates an overview of the model switching system identifying and installing an indicated version of a variant analysis model for executing a genomic analysis application in accordance with one or more embodiments.

FIG. 3 illustrates an example flow of the model switching system installing an indicated version of a variant analysis model utilizing a container orchestration engine in accordance with one or more embodiments.

FIG. 4 illustrates an example flow of the model switching system installing versions of a variant analysis model on a genomic analysis device in accordance with one or more embodiments.

FIG. 5 illustrates an example flow of the model switching system installing different versions of a variant analysis model for different workflow pods in accordance with one or more embodiments.

FIG. 6 illustrates an example architecture diagram of the model switching system in relation to an overall sequencing environment in accordance with one or more embodiments.

FIG. 7 illustrates a flowchart of a series of acts for identifying and installing an indicated version of a variant analysis model for executing a genomic analysis application in accordance with one or more embodiments.

FIG. 8 illustrates a block diagram of an example computing device for implementing one or more embodiments of the present disclosure.

FIG. 9 illustrates a block diagram of an example optical system for image-based genomic sequencing in accordance with one or more embodiments.

FIG. 10 illustrates an example imager for image-based genomic sequencing in accordance with one or more embodiments.

FIG. 11 illustrates an example diagram for performing image-based genomic sequencing in accordance with one or more embodiments.

### DETAILED DESCRIPTION

This disclosure describes embodiments of a model switching system that update a genomic analysis device by installing an indicated version of a variant analysis model required or otherwise indicated by a genomic analysis application. In practical scenarios, a genomic analysis application often requires coordination with, or operation on, a genomic analysis device, such as a field programmable gate array (FPGA) or a central processing unit (CPU), to execute its sequencing, secondary analysis, and/or tertiary analysis functions. Consequently, a genomic analysis application often requires or calls specific functions of a variant analysis model installed on the genomic analysis device, where certain (e.g., older or newer) versions of the variant analysis model include different functions (and different modifications to existing functions). Accordingly, the model switching system can analyze an application specification (e.g., workflow pod specification) defining parameters for a genomic analysis application to determine an indicated version of the variant analysis model (e.g., a version required for executing the genomic analysis application).

Based on determining the indicated version of the variant analysis model, the model switching system can further install the indicated version on a genomic analysis device. In some cases, the model switching system replaces a previously installed version of the variant analysis model, while in other cases the model switching system installs the indicated version in addition to one or more previously installed versions. The model switching system can further execute the genomic analysis application to analyze nucleotide base calls utilizing the indicated version of the variant analysis model.

As just mentioned, in certain implementations, the model switching system identifies a genomic analysis application for analyzing nucleotide base calls determined for a sample nucleotide sequence. For example, the model switching system identifies a genomic analysis application that performs a particular diagnostic (e.g., to detect a marker for a genetic condition) or some other sequencing task or secondary analysis or tertiary analysis on nucleotide base calls. In addition, the model switching system can determine compatibility requirements for the genomic analysis application, including an indicated version of a variant analysis model for executing the genomic analysis application. For instance, the model switching system analyzes an application specification (e.g., workflow pod specification) that defines parameters for the genomic analysis application, including an indicated version of a variant analysis model required for performing one or more functions of the genomic analysis application.

Based on determining the indicated version of the variant analysis model, the model switching system can further install the indicated version on a genomic analysis device, such as an FPGA or a CPU, that implements the variant analysis model and/or various components or functions of the genomic analysis application. For example, the model switching system can update the genomic analysis device to install the indicated version of the variant analysis model to be compatible with the genomic analysis application. In some embodiments, a genomic analysis device can be configured to execute only a single version of a variant analysis model installed at a time (e.g., where the genomic analysis device is an FPGA with hardware can only operate according to its programming). Thus, in these or other embodiments, the model switching system can install the indicated version of the variant analysis model to replace a previously installed version. In certain embodiments, the genomic analysis device may be capable of including multiple versions of the variant analysis model at once, so the model switching system can install an indicated version for a specific genomic analysis application while retaining one or more previously installed versions on the genomic analysis device.

As further mentioned above, the model switching system can also execute a genomic analysis application. In particular, the model switching system can execute a genomic analysis application to perform a particular diagnostic or some other analysis on nucleotide base calls for a sample nucleotide sequence. To execute the genomic analysis application, the model switching system can utilize an indicated version of a variant analysis model installed on a genomic analysis device. Indeed, in some cases, the model switching system executes one or more functions of the genomic analysis application utilizing the genomic analysis device (with the indicated version of the variant analysis model) and/or executes one or more functions that utilize data (e.g., sequencing data, such as nucleotide base calls) in a specific format generated by the indicated version of the variant analysis model.

As suggested above, embodiments of the model switching system provide several advantages, benefits, and/or improvements over existing sequencing systems. For instance, in some embodiments, the model switching system utilizes or implements a new functionality not found in existing sequencing systems. Specifically, the model switching system can automatically identify and install indicated versions of a variant analysis model specific to individual genomic analysis applications or workflow pods. By contrast, existing sequencing systems cannot automatically adapt to applications that require different versions of a variant analysis model. Indeed, some existing sequencing systems instead require a client device to input computer code command lines to change or update a version of a variant analysis model. Such commands often require root access, which many client devices or end users do not have for a variant analysis model.

Utilizing this new functionality not found in existing sequencing systems, in some embodiments, the model switching system is more flexible than many existing sequencing systems. While many existing systems are limited to a specific version of a genomic analysis model and cannot automatically adapt to install new or different versions, the model switching system can automatically update versions of a variant analysis model installed on a genomic analysis device (e.g., on an application-by-application basis). Indeed, the model switching system utilizes a flexible architecture, including a container orchestration engine, that facilitates greater adaptability for updating hardware and/or software components of a genomic analysis device for executing a genomic analysis application. As a result, the model switching system can further prevent or overcome some compatibility issues prevalent in existing sequencing systems that cannot execute certain applications due to the limitations of their variant analysis models or computing devices executing such variant analysis models.

The model switching system can further exhibit improved flexibility in adapting to different genomic analysis applications. To elaborate, the model switching system can determine application-specific (or pod-specific) versions of a variant analysis model indicated by respective applications (or workflow pods) that are to be executed in sequence. The model switching system can further perform an automated process, requiring no user input specific to installation, to iteratively: i) determine the indicated version of the variant analysis model for each application (or workflow pod), ii) install in the indicated version for a particular application (or workflow pod), iii) execute the application utilizing the indicated version, and iv) repeat the process of steps i) through iii) for subsequent applications (or workflow pods), updating the version of the variant analysis model on the genomic analysis device as necessary.

Due at least in part to its increased flexibility, in certain embodiments, the model switching system improves computational efficiency and speed over existing sequencing systems as well. For instance, rather than wasting computational resources, such as processing power and memory, in modifying or retraining model architectures, as required by some existing sequencing systems, the model switching system utilizes a container orchestration engine that facilitates quick, automatic updates and installation of model versions. Along these lines, the model switching system further improves speed over existing sequencing systems by preventing or circumventing the need for constant modifying or retraining model architectures (or waiting for another party with root access to perform an update to install a compatible version of a variant analysis model). Indeed, the model switching system exhibits fast performance in automatically (e.g., without specific user input for determining model version) and seamlessly determining an indicated version of a variant analysis model and automatically (e.g., without specific user input for installation or version switching) and seamlessly installing the indicated version on a genomic analysis device. In some implementations, the process of identifying

As suggested by the foregoing discussion, this disclosure utilizes a variety of terms to describe features and benefits of the model switching system. Additional detail is hereafter provided regarding the meaning of these terms as used in this disclosure. As used in this disclosure, for instance, the term “sample nucleotide sequence” or “sample sequence” refers to a sequence of nucleotides isolated or extracted from a sample organism (or a copy of such an isolated or extracted sequence). In particular, a sample nucleotide sequence includes a segment of a nucleic acid polymer that is isolated or extracted from a sample organism and composed of nitrogenous heterocyclic bases. For example, a sample nucleotide sequence can include a segment of deoxyribonucleic acid (DNA), ribonucleic acid (RNA), or other polymeric forms of nucleic acids or chimeric or hybrid forms of nucleic acids noted below. More specifically, in some cases, the sample nucleotide sequence is found in a sample prepared or isolated by a kit and received by a sequencing device.

As further used herein, the term “sequencing data” refers to data or information pertaining to a nucleotide sequence for one or more genomic samples. For example, sequencing data can include data generated by a sequencing device and/or a variant analysis model. In some cases, sequencing data includes nucleotide reads, nucleotide base calls, and/or sequencing metrics associated with a sample nucleotide sequence. In one or more embodiments, sequencing data is specific to a particular nucleotide sequence and is generated using proprietary methods and processes of a genomic analysis platform that includes a variant analysis model implemented by a genomic sequence processing device.

Relatedly, the term “nucleotide base call” (or sometimes simply “call”) refers to a determination or prediction of a particular nucleotide base (or nucleotide base pair) for a genomic coordinate of a sample genome or for an oligonucleotide during a sequencing cycle. In particular, a nucleotide base call can indicate (i) a determination or prediction of the type of nucleotide base that has been incorporated within an oligonucleotide on a nucleotide-sample slide (e.g., read-based nucleotide base calls) or (ii) a determination or prediction of the type of nucleotide base that is present at a genomic coordinate or region within a sample genome, including a variant call or a non-variant call in a digital output file. In some cases, for a nucleotide read, a nucleotide base call includes a determination or a prediction of a nucleotide base based on intensity values resulting from fluorescent-tagged nucleotides added to an oligonucleotide of a nucleotide-sample slide (e.g., in a well of a flow cell). Alternatively, a nucleotide base call includes a determination or a prediction of a nucleotide base to chromatogram peaks or electrical current changes resulting from nucleotides passing through a nanopore of a nucleotide-sample slide. By contrast, a nucleotide base call can also include an initial or final prediction of a nucleotide base at a genomic coordinate of a sample genome for a variant call file or other base call output file-based on nucleotide reads corresponding to the genomic coordinate. Accordingly, a nucleotide base call can include a base call corresponding to a genomic coordinate and a reference genome, such as an indication of a variant or a non-variant at a particular location corresponding to the reference genome. Indeed, a nucleotide base call can refer to a variant call, including but not limited to, a single nucleotide polymorphism (SNP), an insertion or a deletion (indel), or base call that is part of a structural variant. By using nucleotide base call, a sequencing system determines a sequence of a nucleic acid polymer. For example, a single nucleotide base call can comprise an adenine call, a cytosine call, a guanine call, or a thymine call for DNA (abbreviated as A, C, G, T) or a uracil call (instead of a thymine call) for RNA (abbreviated as U).

As used herein, the term “sequencing metric” refers to a quantitative measurement or score indicating a degree to which an individual nucleotide base call (or a sequence of nucleotide base calls) aligns, compares, or quantifies with respect to a genomic coordinate or genomic region of a reference genome, with respect to nucleotide base calls from nucleotide reads, or with respect to genomic sequencing or genomic structure. For instance, a sequencing metric includes a quantitative measurement or score indicating a degree to which (i) individual nucleotide base calls align, map, or cover a genomic coordinate or reference base of a reference genome; (ii) nucleotide base calls compare to reference or alternative nucleotide reads in terms of mapping, mismatch, base call quality, or other raw sequencing metrics; or (iii) genomic coordinates or regions corresponding to nucleotide base calls demonstrate mappability, repetitive base call content, DNA structure, or other generalized metrics.

Relatedly, as used herein, the term “nucleotide read” (or sometimes simply “read”) refers to an inferred sequence of one or more nucleotide bases (or nucleotide base pairs) from all or part of a sample nucleotide sequence. In particular, a nucleotide read includes a determined or predicted sequence of nucleotide base calls for a nucleotide fragment (or group of monoclonal nucleotide fragments) from a sequencing library corresponding to a genome sample. For example, the model switching system determines a nucleotide read by generating nucleotide base calls for nucleotide bases passed through a nanopore of a nucleotide-sample slide, determined via fluorescent tagging, or determined from a well in a flow cell.

As noted, in some embodiments, the model switching system utilizes a variant analysis model to generate a nucleotide base call for a genomic coordinate and/or to perform other analysis in relation to nucleotide base calls. As used herein, the term “variant analysis model” refers to a model including an algorithm or a set of algorithms for analyzing data for a sample nucleotide sequence (e.g., base call data). In some cases, a variant analysis model is a probabilistic model that generates sequencing data from nucleotide reads of a sample nucleotide sequence, including nucleotide base calls (e.g., variant calls) and associated metrics (e.g., base call quality metrics). For example, in some cases, a variant analysis model refers to a Bayesian probability model that generates variant calls based on nucleotide reads of a sample nucleotide sequence. Such a model can include a model for secondary analysis performed by a server executing variant-call software to align samples' nucleotide reads with a reference genome, determine genetic variants of samples based on the aligned nucleotide reads with respect to the reference genome, and determine one or more of quality metrics, allele frequency metrics, or other sequencing metrics. A variant analysis model may likewise include multiple components, including, but not limited to, different software applications or components for mapping and aligning, sorting, duplicate marking, computing read pileup depths, and variant calling. In some cases, the variant analysis model refers to the ILLUMINA DRAGEN model for variant calling functions and mapping and alignment functions.

As mentioned, in some embodiments, the model switching system utilizes a container orchestration engine to orchestrate execution of genomic analysis application. As used herein, the term “container orchestration engine” refers to a software engine or platform for automating deployment, scaling, and management of containerized software services and applications. For example, a container orchestration engine can include a software application having a microservice architecture that executes individual workflow containers as part of a genomic analysis application (e.g., a sequencing diagnostic workflow). The container orchestration engine can treat each container separately for performing discrete functionalities (e.g., containerized tasks) that can be compartmentalized and added or removed from workflows in a piecewise fashion.

Relatedly, the term “workflow container” (or sometimes simply “container”) refers to a unit of software that packages code (and all its dependencies) for portable deployment. For example, a workflow container includes a compartmentalized or containerized, manipulable, moveable, and executable body of code that performs a particular function or task. In some cases, a workflow container is executable to perform a function or task (e.g., a process or thread) to, for instance, generate a particular output from a piece of sequencing data (e.g., a final output or an intermediate output that feeds into another container). The model switching system can treat workflow containers separately, isolating some containers differently than others to permit and/or prevent access to sequencing data in a specific and tailored fashion (e.g., within one or more workflow data sources). In some cases, a container refers to a NEXTFLOW container and/or a KUBERNETES container.

Relatedly, the term “workflow pod” can refer to a deployable unit of software within a container orchestration engine that includes a group of one or more workflow containers. In some cases, a workflow pod is the smallest deployable unit or denomination of software that a container orchestration engine can execute. Within a workflow pod, the constituent containers can share a common network and/or common computing resources, such as storage locations and processing devices (e.g., a genomic analysis device). In some cases, a workflow pod is part of a genomic analysis application.

As mentioned, in some embodiments, the model switching system determines an indicated version of a variant analysis model for executing a genomic analysis application. As used herein, the term “genomic analysis application” can refer to a package of custom workflow content deployable by the model switching system and that includes workflow definitions, containerized tasks, custom user interface, custom microservices, and/or reference data. A genomic analysis application can include or define a workflow or a collection of tasks or functions organized and orchestrated together to product a set of diagnostic outputs from sequencing data for a sample nucleotide sequence. For example, a workflow of a genomic analysis application may comprise any number of tasks that provide any number of functions, which may include secondary analysis, tertiary analysis, custom QC logic, reporting, or other desired functionality. In some cases, multiple entities can develop a single genomic analysis application that can be deployed locally (e.g., at an edge server) or in the cloud. A genomic analysis application can have a particular structure or file type (e.g., tape archive or “TAR” files) that define workflows and other application data. In some embodiments, a genomic analysis application is a single deployable unit that can be installed on a system that is completely disconnected from the Internet. An application package can be signed to ensure authenticity and validity. The package can be uploaded to a server via a UI portal (e.g., using a browser).

As mentioned, in some embodiments, the model switching system installs an indicated version of a variant analysis model on a genomic analysis device. As used herein, the term “genomic analysis device” can refer to a processing device that executes functions or tasks of a variant analysis model or a genomic analysis application to perform an analysis on sequencing data, such as nucleotide base calls. For example, a genomic analysis device can execute all or part of a genomic analysis application utilizing a variant analysis model to perform a diagnostic analysis or some other analysis on sequencing data. In some cases, a genomic analysis device can include a computational hardware device, such as an FPGA that includes an array of programmable logic blocks for executing functions according to a hardware description language (HDL). As another example, a genomic analysis device can include programmable circuitry for executing instructions that make up a software application or program, such as a genomic analysis application. In some embodiments, a genomic analysis device operates on a server separate from a sequencing device or instrument. In these or other embodiments, the genomic analysis device is housed on a server that shares a local network with a sequencing device.

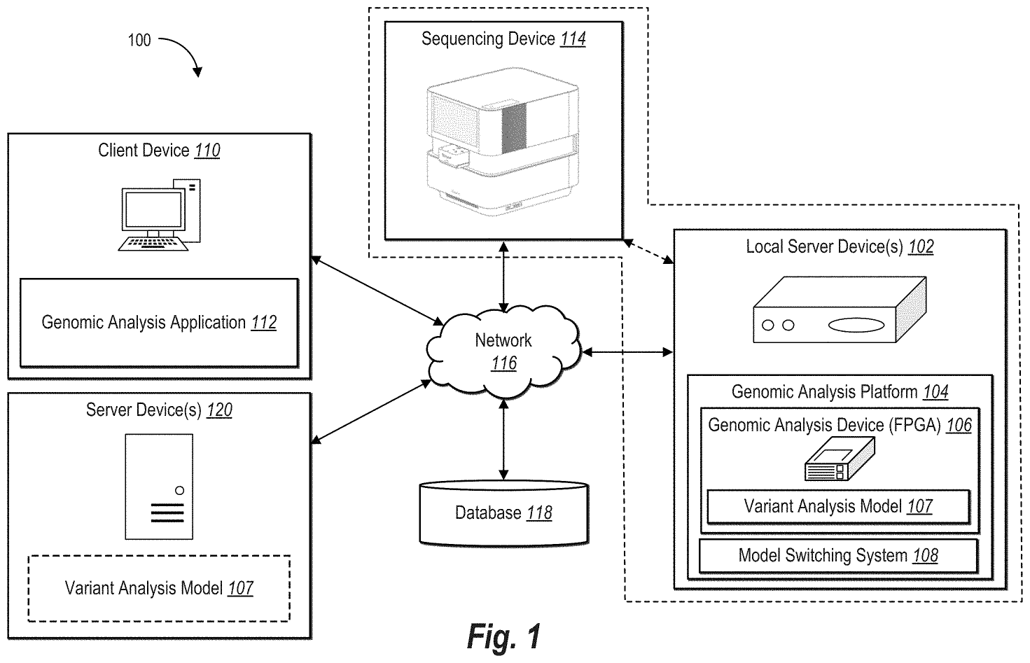

The following paragraphs describe the model switching system with respect to illustrative figures that portray example embodiments and implementations. For example, FIG. 1 illustrates a schematic diagram of a system environment (or “environment”) 100 in which a model switching system 108 operates in accordance with one or more embodiments. As illustrated, the environment 100 includes one or more local server device(s) 102 connected to a client device 110, remote (e.g., cloud) server device(s) 120, and a sequencing device 114 via a network 116. While FIG. 1 shows an embodiment of the model switching system 108, this disclosure describes alternative embodiments and configurations below.

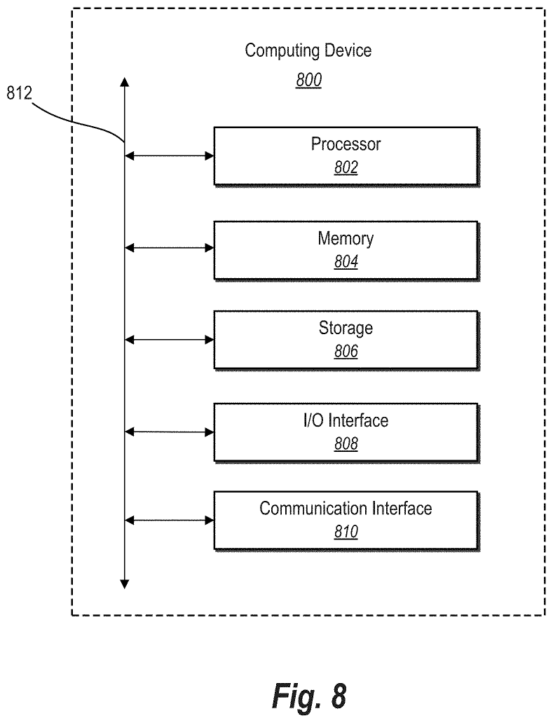

As shown in FIG. 1, the local server device(s) 102, the client device 110, the server device(s) 120, and the sequencing device 114 can communicate with each other via the network 116. The network 116 comprises any suitable network over which computing devices can communicate. Example networks are discussed in additional detail below with respect to FIG. 8.

As indicated by FIG. 1, the sequencing device 114 comprises a device for sequencing a nucleic acid polymer. In some embodiments, the sequencing device 114 analyzes nucleic acid segments or oligonucleotides extracted from samples to generate nucleotide reads or other data utilizing computer implemented methods and systems (described herein) either directly or indirectly on the sequencing device 114. More particularly, the sequencing device 114 receives and analyzes, within nucleotide-sample slides (e.g., flow cells), nucleic acid sequences extracted from samples. In one or more embodiments, the sequencing device 114 utilizes sequencing-by-synthesis (SBS) to sequence nucleic acid polymers into nucleotide reads. In addition or in the alternative to communicating across the network 116, in some embodiments, the sequencing device 114 bypasses the network 116 and communicates directly with the local server device(s) 102 (and/or the client device 110). Indeed, in some embodiments, the sequencing device 114 and the local server device(s) 102 share a local network (e.g., housed on the same or different servers), as indicated by the dashed box, while the client device 110 does not share the local network and instead communicates via the network 116.

As further indicated by FIG. 1, the local server device(s) 102 may generate, receive, analyze, store, and transmit digital data, such as data for determining nucleotide base calls, sequencing nucleic acid polymers, and/or executing a genomic analysis application for analyzing (e.g., performing diagnostics on) a nucleotide sequence. As shown in FIG. 1, the sequencing device 114 may send (and the local server device(s) 102 may receive) call data from the sequencing device 114. The local server device(s) 102 may also communicate with the client device 110. In particular, the local server device(s) 102 can send data to the client device 110, including a variant call file or other information indicating nucleotide base calls, sequencing metrics, error data, diagnostic information, or results generated by a executing a genomic analysis application.

In some embodiments, the serve device(s) 102 comprise a local server device that is located at or near a same physical location of the sequencing device 114. Indeed, in some embodiments, the serve device(s) 102 and the sequencing device 114 are integrated into a same computing device, as indicated by dotted lines around the local server device(s) 102 and the sequencing device 114.

Rather than placed locally with the sequencing device 114, in some embodiments, the local server device(s) 102 comprise a distributed collection of servers where the local server device(s) 102 include a number of server devices distributed across the network 116 and located in the same or different physical locations. As suggested, in some cases, the local server device(s) 102 house the genomic analysis platform 104. Further, the local server device(s) 102 can comprise a content server, an application server, a communication server, a web-hosting server, or another type of server.

As further shown in FIG. 1, the local server device(s) 102 can include a genomic analysis platform 104 for generating and analyzing sequencing data. Generally, the genomic analysis platform 104 includes a genomic analysis device 106 such as an FPGA or a CPU that houses and executes a variant analysis model 107. Indeed, the variant analysis model 107 can generate and/or analyzes sequencing data, such as nucleotide base calls and/or sequencing metrics received from the sequencing device 114, to determine nucleotide base sequences for nucleic acid polymers. For example, the variant analysis model 107 can receive raw data from the sequencing device 114 and can determine a nucleotide base sequence for a nucleic acid segment. In some embodiments, the variant analysis model 107 determines the sequences of nucleotide bases in DNA and/or RNA segments or oligonucleotides. In addition to processing and determining sequences for nucleic acid polymers, the variant analysis model 107 also generates a variant call file indicating one or more nucleotide base calls and/or variant calls for one or more genomic coordinates.

Further, in one or more embodiments, the genomic analysis platform 104 includes the model switching system 108. As just mentioned, the model switching system 108 determines and installs versions of the variant analysis model 107 on the genomic analysis device 106. For example, the model switching system 108 determines that a previously installed version of the variant analysis model 107 is incompatible with a genomic analysis application and updates the genomic analysis device 106 to include a compatible version. In some cases, the model switching system 108 determines an indicated version of the variant analysis model 107 from a genomic analysis application and installs the indicated version on the genomic analysis device 106.

Indeed, in some embodiments, the model switching system 108 identifies or receives a genomic analysis application 112. For example, the model switching system 108 receives the genomic analysis application 112 via upload on a web interface through the network 116. The genomic analysis application 112 can include a number of workflow pods for performing tasks or functions in relation to sequencing data (e.g., for diagnostics). Indeed, the model switching system 108 can execute the genomic analysis application 112 by utilizing an installed version of the variant analysis model 107 (on the genomic analysis device 106) to analyze sequencing data, such as nucleotide base calls and/or sequencing metrics (e.g., from the sequencing device 114 or the variant analysis model 107) to generate or determine various diagnostics or other types of genomic analysis. For example, the model switching system 108 performs a diagnostic analysis of sequencing data for a sample nucleotide sequence. In some cases, the model switching system 108 performs a diagnostic analysis to diagnose, or determine propensities for, one or more diseases or genetic conditions.

As further illustrated in FIG. 1, the client device 110 can generate, store, receive, and send digital data. In particular, the client device 110 can receive sequencing metrics from the sequencing device 114. Furthermore, the client device 110 may communicate with the local server device(s) 102 to receive results of a genomic analysis application and/or a variant call file comprising nucleotide base calls and/or other metrics, such as a call-quality, a genotype indication, and a genotype quality. The client device 110 can accordingly present or display information pertaining to the nucleotide base call within a graphical user interface to a user associated with the client device 110. In addition, the client device 110 can generate and provide (e.g., upload via the network 116) the genomic analysis application 112 that includes one or more workflow pods for performing an analysis of sequencing data. Indeed, the client device 110 can receive, via a graphical user interface, user interaction selecting and arranging or organizing workflow pods and/or containers for to generate the genomic analysis application 112. The client device 110 can also receive a result of the genomic analysis application 112 from the local server device(s) 102 and can display the result within a graphical user interface.

The client device 110 illustrated in FIG. 1 may comprise various types of client devices. For example, in some embodiments, the client device 110 includes non-mobile devices, such as desktop computers or servers, or other types of client devices. In yet other embodiments, the client device 110 includes mobile devices, such as laptops, tablets, mobile telephones, or smartphones. Additional details regarding the client device 110 are discussed below with respect to FIG. 8.

As mentioned, the client device 110 includes a genomic analysis application 112. The genomic analysis application 112 may be a web application or a native application stored and executed on the client device 110 (e.g., a mobile application, desktop application). The genomic analysis application 112 can include instructions that (when executed) cause the client device 110 to receive data from the model switching system 108 and present, for display at the client device 110, data from a variant call file. Furthermore, the genomic analysis application 112 can instruct the client device 110 to display a visualization of the genomic analysis application 112 and/or workflow pods/containers to arrange within the external sequencing diagnostic workflow, as well as a diagnostic result received from the server device(s) upon executing the genomic analysis application 112.

In some embodiments, the model switching system 108 may be located on the client device 110 as part of the genomic analysis application 112 or on the sequencing device 114. Accordingly, in some embodiments, the model switching system 108 is implemented by (e.g., located entirely or in part) on the client device 110. In yet other embodiments, the model switching system 108 is implemented by one or more other components of the environment 100, such as the sequencing device 114. In particular, the model switching system 108 can be implemented in a variety of different ways across the local server device(s) 102, the server device(s) 120, the network 116, the client device 110, and the sequencing device 114. For example, the model switching system 108 can be downloaded from the local server device(s) 102 and/or the server device(s) 120 to the client device 110 and/or to the sequencing device 114 where all or part of the functionality of the model switching system 108 is performed at each respective device within the environment 100.

As further illustrated in FIG. 1, the environment 100 includes a database 118. The database 118 can store information such as genomic analysis applications, application results, variant call files, sample nucleotide sequences, and sequencing data such as nucleotide reads, nucleotide base calls, variant calls, and sequencing metrics. In some embodiments, the local server device(s) 102, the client device 110, and/or the sequencing device 114 communicate with the database 118 (e.g., via the network 116) to store and/or access information, such as genomic analysis applications, application results, variant call files, sample nucleotide sequences, and sequencing data such as nucleotide reads, nucleotide base calls, variant calls, and sequencing metrics. In some cases, the database 118 also stores one or more models, such as different versions of the variant analysis model 107.

Additionally, the environment 100 includes server device(s) 120. In some embodiments, the server device(s) 120 may generate, receive, analyze, store, and transmit digital data, such as data for determining nucleotide base calls, sequencing nucleic acid polymers, and/or executing a genomic analysis application for analyzing (e.g., performing diagnostics on) a nucleotide sequence. In some cases, the sequencing device 114 may send (and the server device(s) 120 may receive) call data from the sequencing device 114. The server device(s) 120 may also communicate with the client device 110. In particular, the server device(s) 120 can send data to the client device 110, including a variant call file or other information indicating nucleotide base calls, sequencing metrics, error data, diagnostic information, or results generated by a executing a genomic analysis application. As shown, the server device(s) 120 can house the variant analysis model 107. In some embodiments, the server device(s) 120 also or alternatively house one or more components of the genomic analysis platform 104 (operating in conjunction with other components on the local server device(s) 102). Further, the server device(s) 120 can comprise a content server, an application server, a communication server, a web-hosting server, or another type of server.

Though FIG. 1 illustrates the components of environment 100 communicating via the network 116, in certain implementations, the components of environment 100 can also communicate directly with each other, bypassing the network 116. For instance, and as previously mentioned, in some implementations, the client device 110 communicates directly with the sequencing device 114. Additionally, in some embodiments, the client device 110 communicates directly with the model switching system 108. Moreover, the model switching system 108 can access one or more databases housed on or accessed by the local server device(s) 102 or the server device(s) 120 or elsewhere in the environment 100.

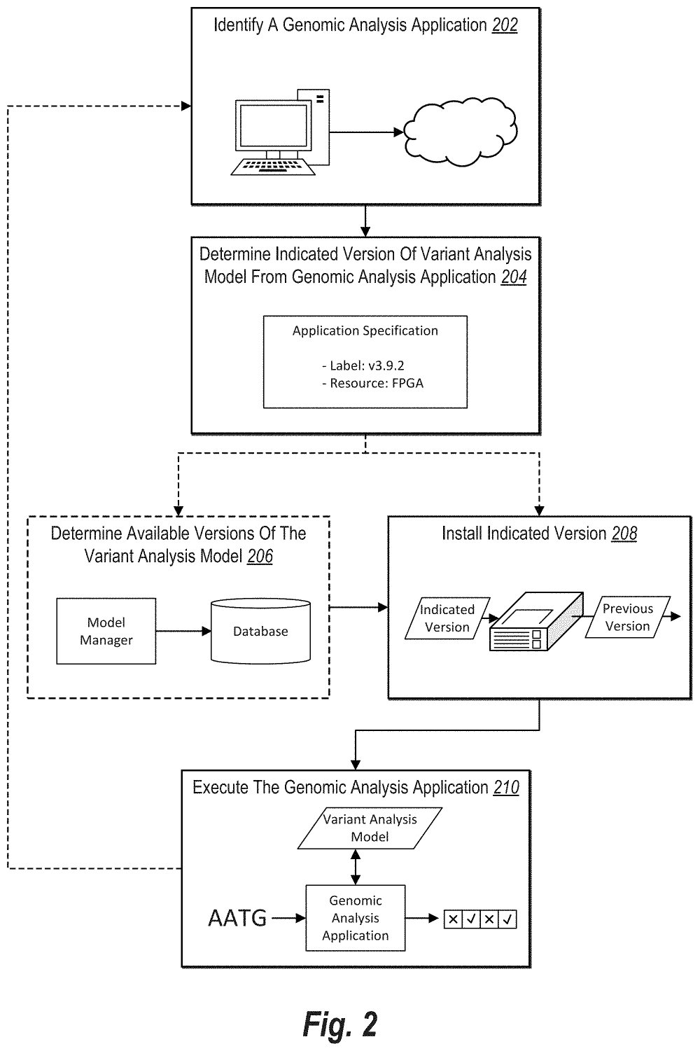

As mentioned, in certain described embodiments, the model switching system 108 installs an indicated version of a variant analysis model for executing a genomic analysis application. In particular, the model switching system 108 identifies an indicated version for a genomic analysis application and installs the version on a genomic analysis device. FIG. 2 illustrates an example overview of installing an indicated version of a variant analysis model for executing a genomic analysis application in accordance with one or more embodiments. Additional detail regarding the individual acts described in relation to FIG. 2 is provided thereafter with reference to subsequent figures.

As illustrated in FIG. 2, the model switching system 108 performs an act 202 to identify a genomic analysis application. In particular, the model switching system 108 identifies a genomic analysis application (e.g., the genomic analysis application 112) for analyzing sequencing data such as nucleotide base calls or sequencing metrics as part of a diagnostic process or for some other purpose. In some cases, the model switching system 108 receives the genomic analysis application from the client device 110 or from a server communicating with the client device 110 (e.g., via the network 116). For example, the model switching system 108 (or the genomic analysis platform 104) provides a web interface whereby the client device 110 generates and uploads a genomic analysis application such as the genomic analysis application 112.

As further illustrated in FIG. 2, the model switching system 108 performs an act 204 to determine an indicated version of a variant analysis model (e.g., the variant analysis model 107) from the genomic analysis application. To elaborate, the model switching system 108 determines a version of a variant analysis model indicated by the genomic analysis application and/or that is required to execute or perform one or functions of the genomic analysis application. In some cases, the model switching system 108 determines the indicated version by analyzing an application specification that defines one or more parameters for executing the genomic analysis application. For instance, the model switching system 108 identifies a version label within the application specification that indicates the version of the variant analysis model required or preferred for executing the genomic analysis application.

For example, an application specification can refer to metadata or software code that accompanies or is part of a genomic analysis application and that defines application parameters (e.g., using labels). Such application parameters can include a version of a variant analysis model required to execute the application (or a particular workflow pod within the application), computing resources such as genomic analysis devices required and/or accessible by the application, definitions for workflow pods/containers, or other application-specific (or pod-specific) information. A single genomic analysis application can include one or more application specifications, where a single application specification can define parameters for an entire application or for a set of one or more workflow pods within an application (while other application specifications define parameters for other workflow pods).

As further illustrated in FIG. 2, in some embodiments, the model switching system 108 performs an act 206 to determine available versions of the variant analysis model. More specifically, based on determining the indicated version of the variant analysis model for executing a genomic analysis application, the model switching system 108 determines whether the indicated version is available. For example, the model switching system 108 utilizes a variant analysis model manager (e.g., a particular container associated with a container orchestration engine implemented by the model switching system 108 or the genomic analysis platform 104) to access a repository of versions of the variant analysis model (e.g., within the database 118). In some embodiments, the model switching system 108 thus determines the available versions of the variant analysis model stored within the repository. The model switching system 108 can further compare the stored versions with the indicated version for the genomic analysis application.

Additionally, as shown in FIG. 2, the model switching system 108 performs an act 208 to install the indicated version of the variant analysis model. In some embodiments, the model switching system 108 performs the act 208 in response to performing the act 206. In other embodiments, the model switching system 108 performs the act 208 in response to the act 204 (e.g., without performing the act 206). The model switching system 108 installs the indicated version to achieve compatibility for executing the genomic analysis application. For instance, the model switching system 108 updates a genomic analysis device (e.g., a device that houses a single version of the variant analysis model at a time or a device that houses multiple versions of the variant analysis model at a time) by installing the indicated version of the variant analysis model. In some cases, the model switching system 108 replaces a previously installed version of the variant analysis model with the indicated version (e.g., so that the indicated version is housed on the genomic analysis device instead of the previous version), while in other cases the model switching system 108 installs the indicated version in addition to one or more previously installed versions.

In certain embodiments, the model switching system 108 performs one or more of the acts 204-208 automatically, i.e., without requiring or receiving user input to specific initiate or perform the acts or tasks. For example, the model switching system 108 automatically determines an indicated version of a variant analysis model upon identifying or receiving the genomic analysis application, without requiring or receiving user input to initiate identifying the indicated version. In addition (or alternatively), the model switching system 108 automatically determines available versions of the variant analysis model within a repository upon determining the indicated version (or upon identifying the genomic analysis application), without requiring or receiving user interaction to initiate the determination of available versions of the variant analysis model. Additionally (or alternatively) still, the model switching system 108 automatically installs the indicated version of the variant analysis model upon determining the available versions (or upon determining the indicated version), without requiring or receiving user input to initiate installing the indicated version.

As further illustrated in FIG. 2, the model switching system 108 performs an act 210 to execute the genomic analysis application. In particular, the model switching system 108 performs the various functions (as defined by workflow pods and/or containers) of the genomic analysis application utilizing the indicated version of the variant analysis model installed on the genomic analysis device. For instance, the model switching system 108 utilizes the genomic analysis device to implement the variant analysis model to provide data for performing (e.g., used by) certain aspects of the genomic analysis application. In some cases, the model switching system 108 can further utilize the genomic analysis device to perform one or more aspects of the genomic analysis application.

In executing the genomic analysis application, the model switching system 108 determines or generates application results that include diagnostics or other information generated or extrapolated from sequencing data such as nucleotide base calls (e.g., “AATG”) of a sample nucleotide sequence. In certain embodiments, the model switching system 108 executes genomic analysis applications in the form of diagnostic applications that satisfy security and analysis standards set by regulatory bodies (e.g., standards set by the United States Food and Drug Administration or some other body) for in vitro diagnostics (IVD). The model switching system 108 can likewise execute applications for investigational use only (IUO) analysis and research use only (RUO) analysis.

As further illustrated in FIG. 2, the model switching system 108 can repeat the acts 202-210 for multiple genomic analysis applications. To elaborate, the model switching system 108 can perform or execute multiple genomic analysis applications in a sequence (e.g., by scheduling computing resources such as an FPGA or CPU). Indeed, for each application in the sequence, the model switching system 108 can perform the acts 202-210 before then moving on to the next application. In some embodiments, the model switching system 108 automatically iterates through the multiple applications, one at a time, without requiring or receiving user input to proceed to the next application and/or without requiring or receiving user input to perform/repeat each of the acts 202-210 for each successive application.

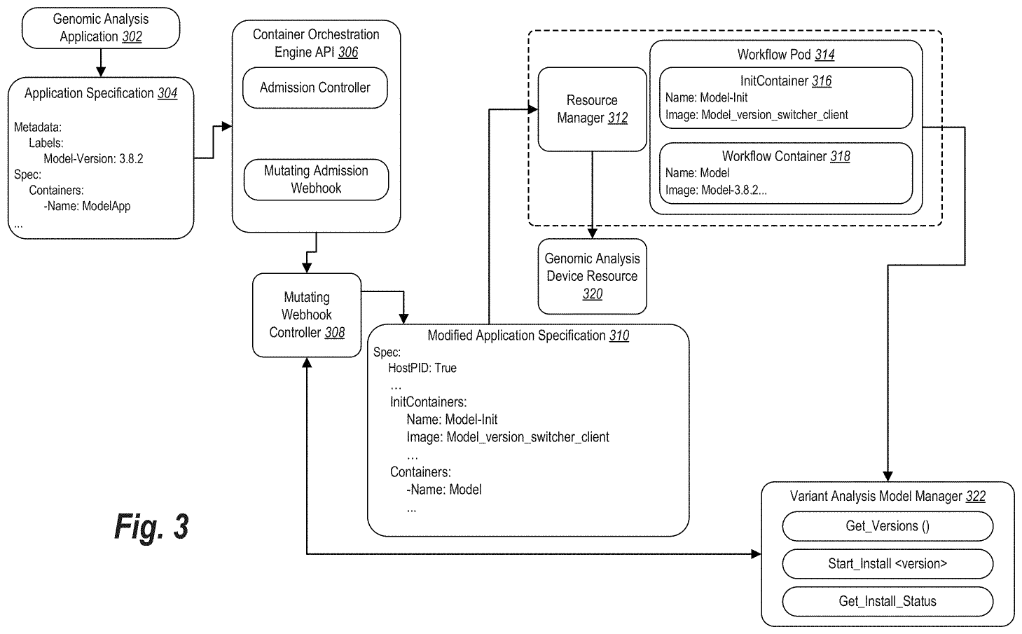

As mentioned, in certain described embodiments, the model switching system 108 utilizes a particular container orchestration engine to switch between versions of a variant analysis model. In particular, the model switching system 108 utilizes a container orchestration engine that executes containerized functions to determine a version of a variant analysis model indicated by a genomic analysis application and to install the indicated version on a genomic analysis device. FIG. 3 illustrates an example flow for utilizing a container orchestration engine to identify and install versions of a variant analysis model in accordance with one or more embodiments.

As illustrated in FIG. 3, the model switching system 108 identifies or receives a genomic analysis application 302 (e.g., the genomic analysis application 112). In addition, the model switching system 108 identifies an application specification 304 within or accompanying the genomic analysis application 302. For example, the model switching system 108 identifies the application specification 304 that defines parameters for executing the genomic analysis application 302, such as version labels specifying the version of a variant analysis model, container labels specifying workflow containers or workflow pods within the genomic analysis application 302, and resource labels specifying computing resources for executing the genomic analysis application 302 (e.g., a genomic analysis device). In some cases, the genomic analysis application 302 includes (or corresponds to) a single application specification (e.g., the application specification 304). In other cases, the genomic analysis application 302 includes (or corresponds to) multiple application specifications, each defining parameters for one or more constituent workflow pods within the genomic analysis application 302.

As further illustrated in FIG. 3, the model switching system 108 utilizes a container orchestration engine API 306 to orchestrate or coordinate identifying and installing indicated versions of a variant analysis model. For example, the model switching system 108 utilizes a container orchestration engine API 306 that includes various workflow pods and/or workflow containers (that are part of workflow pods) for analyzing the application specification 304, identifying indicated versions of variant analysis model, installing the indicated version, and executing a genomic analysis application utilizing the indicated version.

In some cases, the container orchestration engine API 306 includes an admission controller that includes functions for installing versions of a variant analysis model. For example, the admission controller can communicate with other components of the container orchestration engine (or the model switching system 108) to provide instructions for installing an indicated version of the variant analysis model. In these or other cases, the container orchestration engine API 306 includes a mutating admission webhook that modifies or augments behavior of the genomic analysis application 302 with custom callbacks (e.g., by modifying the application specification 304). For instance, the mutating admission webhook provides instructions to, or includes instructions accessible by, the mutating webhook controller 308 to modify or augment the application specification 304.

Indeed, the model switching system 108 utilizes the mutating webhook controller 308 (e.g., as a workflow pod within the container orchestration engine) to generate the modified application specification 310 from the application specification 304. Indeed, the mutating webhook controller 308 can include computer code that keys off a pod label (e.g., an indication of variant analysis model version) to modify the application specification 304 to include pod settings for a version of a variant analysis model (e.g., hostPID and hostIPC). Additionally, the mutating webhook controller 308 includes code to instigate injection of an initialization workflow container, which queries the variant analysis model manager 322 to install a model version indicated by a pod label. Accordingly, the mutating webhook controller 308 ensures that the version of the variant analysis model 107 installed on an operating system matches the version running in the pod corresponding to the pod label.

For example, the model switching system 108 generates the modified application specification 310 to include a specialized workflow container called an initialization workflow container (represented in FIG. 3 as “InitContainer”). Indeed, the model switching system 108 generates and adds the initialization workflow container to the modified application specification 310. In certain cases, the initialization workflow container includes instructions for communicating with other components (e.g., other workflow containers, devices, or network locations) of the model switching system 108 (or the genomic analysis platform 104). For instance, the model switching system 108 utilizes the initialization workflow container to communicate with a variant analysis model manager to install an indicated version of a variant analysis model.

Indeed, the model switching system 108 utilizes the mutating webhook controller 308 to add version labels to the modified application specification 310 for versions of the variant analysis model required or preferred for executing the genomic analysis application 302. The model switching system 108 also utilizes the mutating webhook controller 308 to add resource labels to the modified application specification 310 indicating resources such as an FPGA or another genomic analysis device required or preferred for executing the genomic analysis application 302.

Thus, the mutating webhook controller 308 identifies and/or selects a genomic analysis device by identifying and/or adding a resource label within the modified application specification 310. As shown, the mutating webhook controller 308 further communicates with the variant analysis model manager 322 to provide instructions for installing the indicated version of the variant analysis model. In one or more implementations, the mutating webhook controller 308 and/or the variant analysis model manager 322 are housed on the same server as the variant analysis model (e.g., the server device(s) 120 or the local server device(s) 102).

In one or more embodiments, the mutating webhook controller 308 identifies or detects any genomic analysis application or workflow pod that requests a variant analysis model and mutates its corresponding application specification (or pod specification) by adding an initialization container for installing an indicated version of the variant analysis model. In some embodiments, the model switching system 108 generates (utilizing the mutating webhook controller 308) a modified application specification for each individual genomic analysis application that requests the variant analysis model and/or generates a modified pod specification for each individual workflow pod that requests the variant analysis model.

As further illustrated in FIG. 3, the model switching system 108 utilizes a resource manager 312 to access or utilize a genomic analysis device resource 320 (as specified by the modified application specification 310). To elaborate, the model switching system 108 utilizes a particular workflow container called a resource manager 312 to identify a resource label within the modified application specification 310 and to access the resource identified in the label. Indeed, the model switching system 108 defines a genomic analysis device such as an FPGA or a CPU as a schedulable resource for access via the container orchestration engine. Thus, the resource manager 312 accesses the genomic analysis device resource 320 (e.g., a workflow container that communicates with and interfaces with a genomic analysis device) associated with the resource indicated in the modified application specification 310. In some cases, the modified application specification 310 indicates an FPGA or a CPU or some other genomic analysis device for executing the genomic analysis application 302 (or a particular workflow pod), and the resource manager 312 therefore accesses or communicates with the specified device (or other resource) for facilitating execution of the genomic analysis application 302 (or a particular workflow pod).

As further illustrated in FIG. 3, the model switching system 108 executes a workflow pod 314 as part of the genomic analysis application 302. In particular, the model switching system 108 identifies a workflow pod 314 associated with the modified application specification 310 and executes functions for one or more workflow containers within the workflow pod 314. Specifically, the model switching system 108 executes functions for the workflow initialization container 316 and the workflow container 318. In certain embodiments, the model switching system 108 executes or performs functions defined by the workflow initialization container 316 before executing functions of other workflow containers (e.g., the workflow container 318) within the workflow pod 314 or within genomic analysis application 302.

Indeed, as described, the model switching system 108 generates and adds the workflow initialization container 316 to the workflow pod 314 to initialize the workflow pod 314 for executing the workflow container 318 utilizing the proper (e.g., indicated) version of the variant analysis model on the requested resource (e.g., genomic analysis device). For instance, in the example illustrated in FIG. 3, the model switching system 108 the workflow initialization container 316 communicates with the variant analysis model manager 322 to install version 3.8.2 of the variant analysis model for executing the workflow container 318. By executing the workflow initialization container 316 before the workflow container 318, the model switching system 108 ensures that the workflow pod 314 utilizes or has access to the proper version of the variant analysis model for performing functions as defined by constituent containers such as the workflow container 318.

As just mentioned, the workflow initialization container 316 communicates with the variant analysis model manager 322 (running on a host operating system) to provide instructions for installing an indicated version of the variant analysis model. Indeed, the variant analysis model manager 322 receives instructions indicating, or otherwise determines, an indicated version of a variant analysis model for executing the workflow pod 314 (or the genomic analysis application 302). The variant analysis model manager 322 further accesses a repository of variant analysis model versions to determine whether the indicated version is available. Upon determining that the indicated version is available, the variant analysis model manager 322 installs the indicated version on a genomic analysis device, either replacing a previously installed version or else adding the indicated version to one or more previously installed versions. The variant analysis model manager 322 can further monitor installation status and provide an indication to the mutating webhook controller 308 and/or to other components of the model switching system 108 to begin executing the genomic analysis application 302 (or the workflow pod 314) upon completion of the installation.

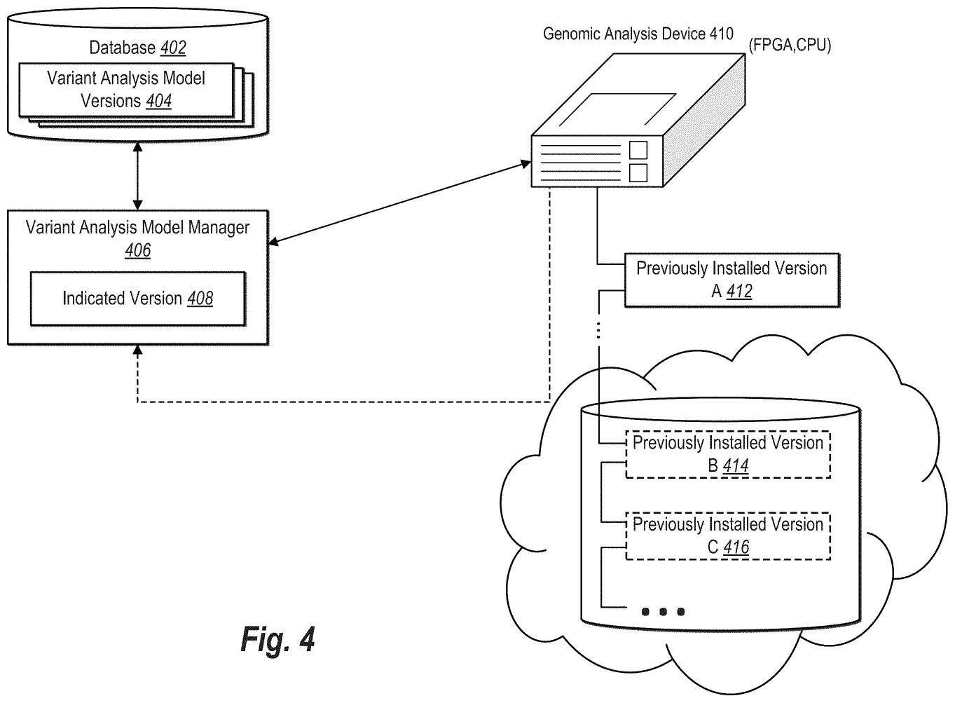

As mentioned above, in certain described embodiments, the model switching system 108 installs a version of a variant analysis model indicated by a genomic analysis application. In particular, the model switching system 108 installs different versions for different genomic analysis applications as requested by the respective applications. FIG. 4 illustrates an example flow for installing indicated versions of a variant analysis model in accordance with one or more embodiments.

As illustrated in FIG. 4, the model switching system 108 utilizes a variant analysis model manager 406 (e.g., the variant analysis model manager 322) to install an indicated version 408 of a variant analysis model on a genomic analysis device 410 (e.g., the genomic analysis device 106. As described, the model switching system 108 determines an indicated version of the variant analysis model and accesses a repository of variant analysis model versions 404 within a database 402 (e.g., the database 118). Additionally, the model switching system 108 utilizes the variant analysis model manager 406 to install the indicated version 408 from the database 402 on the genomic analysis device 410.

In some cases, the database 402 is local to the genomic analysis device 410 and/or the variant analysis model (e.g., on the same server or within a local network), while in other cases the database 402 is remote (e.g., not on the same server or within a local network). Thus, the model switching system 108 can provide instructions to a server (e.g., a server housing or interfaced with the genomic analysis device 410) to install the indicated version 408 based on determining that the indicated version is stored within a remote repository.

In some embodiments, the variant analysis model manager 406 installs the indicated version 408 to replace a previously installed version of the variant analysis model (e.g., the previously installed version A 412). For example, the variant analysis model manager 406 determines that the indicated version 408 is different from a previously installed version of the variant analysis model and therefore installs the indicated version 408. In certain cases, the genomic analysis device 410 is capable of housing only a single version of the variant analysis model at a time. Thus, the variant analysis model manager 406 replaces the previously installed version A 412 with the indicated version 408.

In addition, the model switching system 108 executes a genomic analysis application (or a workflow pod of a genomic analysis application) by implementing the indicated version 408 of the variant analysis model on the genomic analysis device 410. For instance, the model switching system 108 determines a computational availability, such as a determination of whether the genomic analysis device 410 is currently performing a task (where the genomic analysis device 410 is limited to a single task at a time). Based on the computational availability, the model switching system 108 further schedules execution of the genomic analysis application either for immediate execution or for later execution (e.g., in line behind one or more other tasks that are queued for the genomic analysis device 410 from the model switching system 108 or from some other component of the genomic analysis platform 104). Indeed, the model switching system 108 can determine computational availability of the genomic analysis device 410 (e.g., utilizing the variant analysis model manager 322) at each step involving the genomic analysis device 410, such as installing an indicated version of a variant analysis model and executing a genomic analysis application or workflow pod utilizing the indicated version.

Upon executing the genomic analysis application, the model switching system 108 can determine that the genomic analysis device 410 is no longer occupied and is again free to schedule for another genomic analysis application. Thus, the model switching system 108 can analyze another genomic analysis application to identify a new indicated version of the variant analysis model (e.g., as specified by an application specification) for the new application. The model switching system 108 can further install the new indicated version (upon determining that the new indicated version is among the available variant analysis model versions 404) and can execute the new genomic analysis application. Further, the model switching system 108 can repeat the process of identifying indicated versions, installing the indicated versions, and executing the genomic analysis applications for a number of genomic analysis application in a sequence.

In one or more embodiments, the genomic analysis device 410 is capable of housing, (or communicating with a database that houses) multiple versions of the variant analysis model. For example, the genomic analysis device 410 can interface with a database (e.g., the database 118) of variant analysis model versions that are installed either locally (e.g., on a shared server device or within a shared local network) or on a cloud server. As shown, the genomic analysis device 410 interfaces with a cloud database that includes previously installed version B 414 and previously installed version C 416. In some cases, the genomic analysis device 410 can execute a genomic analysis application by accessing the previously installed version B 414 and/or the previously installed version C 416 to perform functions for one or more workflow pods defined within the genomic analysis application. In one or more cases, the genomic analysis device 410 can interface with the cloud database (or a local database or some other local memory) to alternate between different installed versions of the variant analysis model (e.g., for executing different genomic analysis applications or workflow pods).

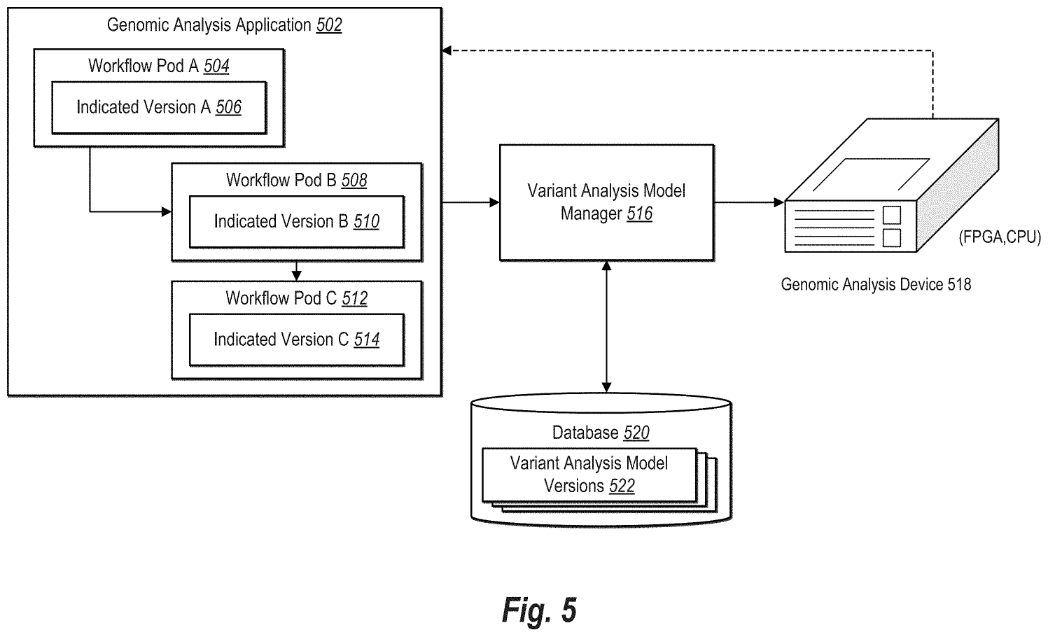

As mentioned, in certain embodiments, the model switching system 108 installs different versions of a variant analysis model for different workflow pods (e.g., within a single genomic analysis application or within different genomic analysis applications). In particular, the model switching system 108 can identify a number of workflow pods within a genomic analysis application and can analyze a single application specification defining different versions of the variant analysis model for executing the respective workflow pods. Or the model switching system 108 can analyze individual application specifications (or pod specifications) defining variant analysis model versions for the individual workflow pods. FIG. 5 illustrates an example flow for installing different versions of a variant analysis model for different workflow pods in accordance with one or more embodiments.

As illustrated in FIG. 5, the model switching system 108 utilizes a variant analysis model manager 516 (e.g., the variant analysis model manager 406 or 322) to identify indicated versions of the variant analysis model and to install the indicated versions on a genomic analysis device 518 (e.g., the genomic analysis device 410 or 106). More specifically, the model switching system 108 analyzes a genomic analysis application 502 to identify a plurality of workflow pods, such as workflow pod A 504, workflow pod B 508, and workflow pod C 512. Additionally, the model switching system 108 identifies one or more application specifications (or pod specifications) that specify or define respective indicated versions for executing the individual workflow pods A-C. In certain cases, the workflow pods A-C indicate different versions of the variant analysis model because their respective genomic analysis functions require different components or functionality belonging to the different versions of the variant analysis model.

For example, the model switching system 108 identifies an indicated version A 506 from an application specification associated with the workflow pod A 504. In addition, the model switching system 108 identifies an indicated version B 510 associated with the workflow pod B 508. Further, the model switching system 108 identifies an indicated version C 514 associated with the workflow pod C 512. In one or more embodiments, the model switching system 108 accesses a database 520 (e.g., the database 118) that includes or stores a repository of variant analysis model versions 522. The model switching system 108 further compares the available variant analysis model versions 522 with the indicated versions A-C (either all at once or sequentially one at a time) to determine whether the indicated versions A-C are available within variant analysis model versions 522.

In some embodiments, the model switching system 108 further utilizes the variant analysis model manager 516 in an iterative, sequential manner to identify and install the indicated versions for each respective workflow pod as they are to be executed via the genomic analysis device 518. To elaborate, the model switching system 108 identifies and installs indicated version A 506 on the genomic analysis device 518 and further utilizes the genomic analysis device 518 to execute the workflow pod A 504 utilizing the indicated version A installed on the genomic analysis device 518. Once execution of workflow pod A 504 is complete, the model switching system 108 continues in series with workflow pod B 508 by identifying the indicated version B 510, verifying its availability among the variant analysis model versions 522, installing it on the genomic analysis device 518, and executing the workflow pod B 508 utilizing the indicated version B.

Likewise, the model switching system 108 repeats the process for workflow pod C 512 by identifying the indicated version C 514, verifying that the indicated version C 514 is available within the variant analysis model versions 522, installing the indicated version C 514 on the genomic analysis device 518, and executing the indicated version C 514 utilizing the indicated version C 514 installed on the genomic analysis device 518. As shown, the model switching system 108 can repeat this process for any number of workflow pods (e.g., workflow pods specifying different indicated versions of a variant analysis model) identified within the genomic analysis application 502.

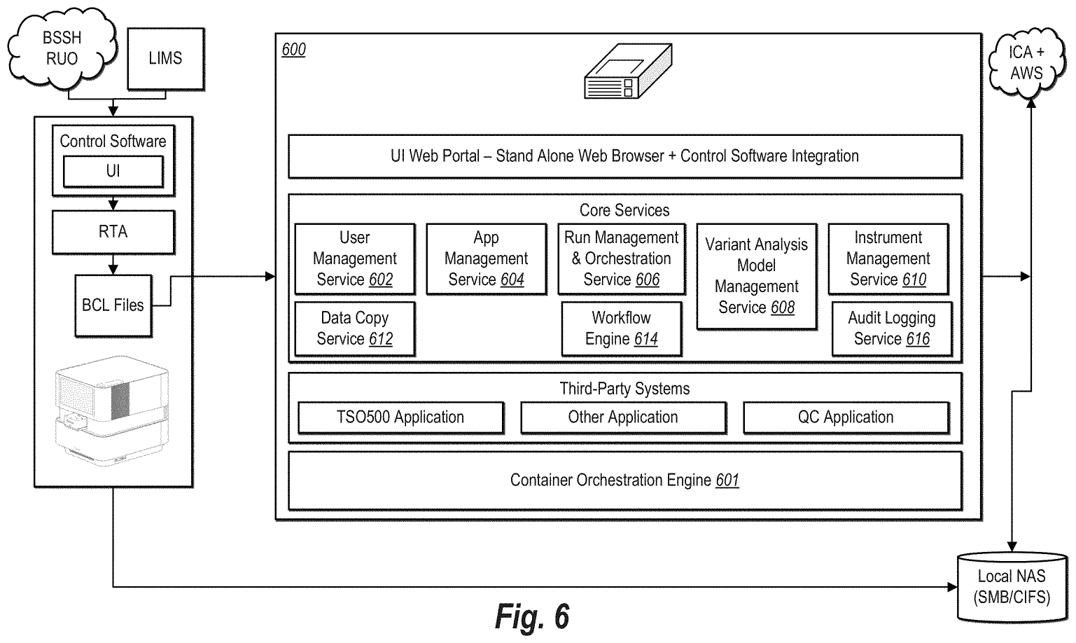

As mentioned above, in certain embodiments, the model switching system 108 utilizes containers and pods to determine and update versions of a variant analysis model. In particular, the model switching system 108 can update a model version based on requirements or parameters for a genomic analysis application. FIG. 6 illustrates an example diagram of components, applications, devices, and containers of a system architecture (e.g., installed on a local server device) involved in updating a model version and executing an application in accordance with one or more embodiments.

As illustrated in FIG. 6, the model switching system 108 utilizes a sequencing device (e.g., the sequencing device 114) to communicate with various components or systems and to perform sequencing operations used by, and/or as instructed by, a version of a variant analysis model on a local server (e.g., the genomic analysis device 106 on the local server device(s) 102). For example, the model switching system 108 communicates with a BaseSpace Sequencing Hub (“BSSH”) or cloud-based interface for research use only (“RUO”) and a lab information management system (“LIMS”) to generate base calls for nucleotide bases of a genomic sample.

Based on information from the BSSH RUO and/or the LIMS, the model switching system 108 performs a real-time analysis (“RTA”) of a sample. More specifically, the model switching system 108 performs RTA to determine base calls, variant calls, and/or various metrics from nucleotide bases of a genomic sample according to a sequencing plan. Based on the RTA, the model switching system 108 generates a binary base call (“BCL”) file that includes raw data generated and output by one or more sequencing runs (e.g., via the RTA). Indeed, the BCL file can indicate base calls, variant calls, and/or other sequencing information for interpretation by a variant analysis model and/or some other system.

To organize or plan a sequencing run of the RTA, the model switching system 108 provides control software (e.g., including a user interface) for planning or scheduling a sequencing run on a particular sample. Indeed, the model switching system 108 provides control software and a user interface for planning one or more sequencing runs to, for example, test a genomic sample for a particular genetic marker or a hereditary trait according to plan parameters. For instance, the control software enables a user to specify parameters for a sequencing run and/or to test for specific markers. As shown, the model switching system 108 can integrate the control software for the sequencing device with a user interface web portal (which includes a standalone web browser and control software integration) to interface with the sequencing device for planning a sequencing run.

In some cases, the model switching system 108 facilitates local planning for a sequencing run, where the planning software (e.g., the control software) is hosted by a local edge server. In these or other cases, the model switching system 108 facilitates cloud planning for a sequencing run, where the planning software (e.g., the control software) is hosted on a cloud server rather than the local server device(s) 102. In a similar fashion, the execution of a variant analysis model can be local or cloud-based as well, depending on whether the server hosting the variant analysis model is a local server (e.g., the local server device(s) 102) or a cloud-based server (e.g., the server device(s) 120).

In one or more embodiments, the model switching system 108 facilitates version updates or version switching for a variant analysis model for dark sites or other locations with computing devices that are not connected to the Internet or Wide Area Network (WAN). For instance, the model switching system 108 accommodates situations where (i) a genomic sequencing system, such as a primary analysis system, a secondary analysis system, or a tertiary analysis system is not connected to the Internet for privacy, security, and/or other purposes and (ii) includes multiple versions of a variant analysis model saved on the relevant system. To elaborate, the model switching system 108 can download an installer (e.g., an executable digital file for installing software on a computing device) for a version of a variant analysis model (or multiple installers for multiple versions of the variant analysis model) and can generate and provide a graphical user interface to facilitate copying the installer onto the model switching system 108 (e.g., on to the genomic analysis device 106). Specifically, the model switching system 108 can guide the installation process using the graphical user interface (e.g., to copy and/or execute an installer) without requiring Internet or WAN access to execute the installer for the relevant or selected model version. For instance, the model switching system 108 can provide multiple installers for different versions of the variant analysis model and can automatically (or manually via input within the graphical user interface) select, copy, and/or execute the appropriate installer for updating or switching between model versions based on parameters or requirements of different genomic analysis applications.

As further illustrated in FIG. 6, the system architecture 600 of the model switching system 108 includes, or communicates with, containers or systems associated with one or more core services. Indeed, as shown, the model switching system 108 includes the services of the system architecture 600. To manage or orchestrate the various services of the system architecture 600 of the model switching system 108, the system architecture 600 includes a container orchestration engine 601 (e.g., K3S or Kubernetes) to manage and implement various pods and containers associated with performing genomic analyses and/or updating model versions for variant analysis models. As described, the model switching system 108 utilizes a container orchestration engine 601 to orchestrate or coordinate identifying and installing indicated versions of a variant analysis model (e.g., as indicated by an application of a third-party system). The model switching system 108 can perform other functions as well, including user management, application management, run management, variant analysis model management, instrument management, data copying, and audit logging.

For example, the model switching system 108 utilizes a container orchestration engine 601 that includes various workflow pods and/or workflow containers (that are part of workflow pods) for analyzing an application specification, identifying indicated versions of variant analysis model, installing the indicated version, and executing a genomic analysis application utilizing the indicated version. In some cases, the container orchestration engine 601 includes an admission controller that includes functions for installing versions of a variant analysis model. For example, the admission controller can communicate with other components of the container orchestration engine 601 (or the model switching system 108) to provide instructions for installing an indicated version of the variant analysis model.

For example, the system architecture 600 includes a user management service 602 (e.g., a set of one or more user management pods or containers). The user management service 602 performs various processes or functions for providing a single sign-on (“SSO”) experience system wide. Specifically, the user management service 602 can include one or more containers or pods that include or access user information for a third-party system to, for example, determine a version of a variant analysis model currently installed on a genomic analysis device and/or to determine a required version of a genomic analysis model. Based on the determination of the installed and/or required versions, the user management service 602 can communicate with other services of the system architecture 600 to initiate installation of a new version to match the required version, as well as removal of the current version.

In addition, the system architecture 600 includes or utilizes an application management service 604 in communication with the container orchestration engine 601. For example, the application management service 604 manages application package installation for version upgrades and removal of old model versions. In some cases, the application management service 604 further includes a resource manager (e.g., the resource manager 312). As described, the resource manager can access or utilize a genomic analysis device resource as specified by the modified application specification. To elaborate, the resource manager identifies a resource label to access a designated resource, such as an FPGA or a CPU, as a schedulable resource for access via the container orchestration engine. In some cases, the application management service 604 includes (or receives from a third-party system) an application specification that indicates an FPGA or a CPU or some other genomic analysis device for executing a genomic analysis application (or a particular workflow pod), and the resource manager therefore accesses or communicates with the specified device (or other resource) for facilitating execution of the genomic analysis application (or the particular workflow pod).

As further shown, the system architecture 600 includes or utilizes a run management and orchestration service 606. To elaborate, the run management and orchestration service 606 includes one or more containers or pods for facilitating and executing genomic analysis, such as a sequencing run, a primary analysis, a secondary analysis, or a tertiary analysis. Indeed, the run management and orchestration service 606 includes computer code or instructions for executing a sequencing run (and/or further analysis) according to an installed version of a variant analysis model. For instance, the run management and orchestration service 606 communicates with the workflow engine 614 to execute a custom workflow for an application, such as an application associated with a third-party system (e.g., an oncology assay application, such as TSO500 application; a QC application; or another application). The run management and orchestration service 606 further includes code for communicating with the data copy service 612 to copy input and output sequencing data (e.g., from a BCL file generated by a sequencing device) for performing a genomic analysis and/or for storing in a database, such as a local network attached storage (“NAS”), server message block (“SMB”), or common internet file system (“CIFS”).

In addition, the system architecture 600 includes a variant analysis model management service 608. In particular, the variant analysis model management service 608 includes one or more containers or pods for managing a variant analysis model (e.g., the variant analysis model 107) for performing genomic analysis. For example, the variant analysis model management service 608 implements a particular version installed to detect a genetic marker for a certain condition within a sample genomic sequence. In addition, the variant analysis model management service 608 manages model peripherals, such as licensing, self-testing, and version authentication for a variant analysis model.

As further illustrated in FIG. 6, the system architecture 600 includes an instrument management service 610. In one or more embodiments, the instrument management service 610 includes one or more containers or pods for pairing and monitoring instruments used as part of a sequencing workflow and/or a genomic analysis workflow after sequencing. For instance, the instrument management service 610 manages instruments of a sequencing device and/or a variant analysis model to pair compatible instruments with indicated versions of a variant analysis model (or vice-versa). The system architecture 600 further includes an audit logging service 616 for monitoring and logging performance of instruments, components of a variant analysis model, and/or containers within an application workflow. For instance, the audit logging service 616 detects and logs errors or other auditing information associated with the system architecture 600.

As suggested above in the description of FIG. 1, using the system architecture 600 illustrated in FIG. 6, the model switching system 108 can be deployed locally on an edge server (e.g., the local server device(s) 102) or in the cloud, such as on cloud-based servers hosting Illumina Connected Analytics (“ICA”) and/or cloud-based servers from Amazon Web Services (“AWS”). For example, the model switching system 108 can be executed locally on the local server device(s) 102 as part of planning software that plans resources based on user input for sequencing runs or other assays, and the variant analysis model 107 can likewise be executed locally on the local server device(s) 102 to analyze BCL data and determine variant calls or other metrics. By contrast, the model switching system 108 can be executed remotely on the server device(s) 120 as part of planning software, and the variant analysis model 107 can be executed locally on the local server device(s) 102 to analyze BCL data and determine variant calls.

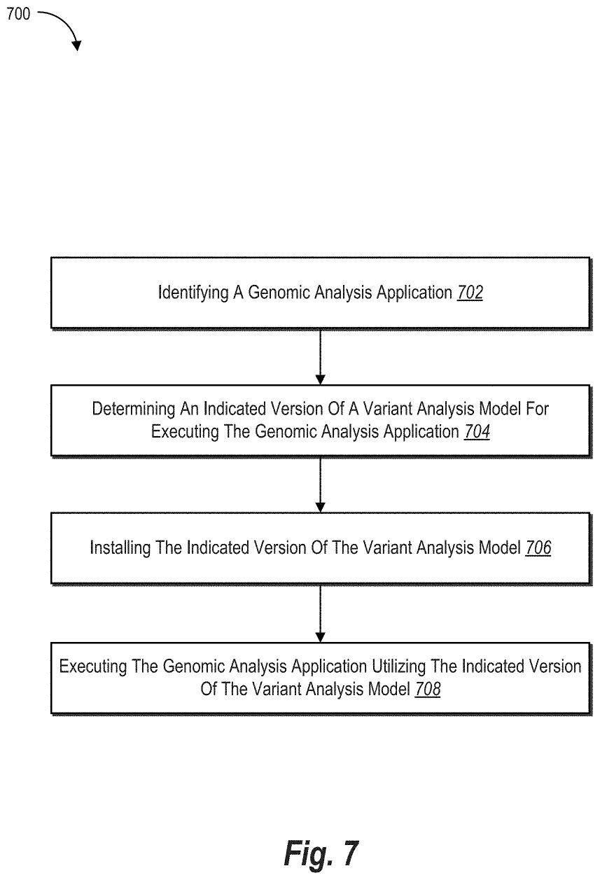

Turning now to FIG. 7, this figure illustrates an example flowchart of a series of acts of identifying and installing an indicated version of a variant analysis model for executing a genomic analysis application in accordance with one or more embodiments. While FIG. 7 illustrates acts according to one embodiment, alternative embodiments may omit, add to, reorder, and/or modify any of the acts shown in FIG. 7. The acts of FIG. 7 can be performed as part of a method. Alternatively, a non-transitory computer readable storage medium can comprise instructions that, when executed by one or more processors, cause a computing device to perform the acts depicted in FIG. 7. In still further embodiments, a system comprising at least one processor and a non-transitory computer readable medium comprising instructions that, when executed by one or more processors, cause the system to perform the acts of FIG. 7.

As shown in FIG. 7, the series of acts 700 includes an act 702 of identifying a genomic analysis application. In particular, the act 702 can include identifying a genomic analysis application for analyzing nucleotide base calls determined for a sample nucleotide sequence. The act 702 can involve identifying or receiving a genomic analysis application from a client device, where the client device arranges or generates the genomic analysis application for upload or transfer to a genomic analysis platform.

As also shown in FIG. 7, the series of acts 700 includes an act 704 of determining an indicated version of a variant analysis model for executing the genomic analysis application. In particular, the act 704 can include determining an indicated version of a variant analysis model for executing the genomic analysis application indicated by an application specification defining one or more parameters for the genomic analysis application. For example, the act 704 can involve analyzing the application specification to identify a version label specifying the indicated version of the variant analysis model. In some cases, the act 704 includes utilizing a variant analysis model manager to analyze the application specification to identify a version label specifying the indicated version of the variant analysis model.

Additionally, the series of acts 700 includes an act 706 of installing the indicated version of the variant analysis model. In particular, the act 706 can include, based on determining the indicated version of the variant analysis model, installing the indicated version of the variant analysis model for execution instead of a previously installed version of the variant analysis model. For example, the act 706 can involve updating a field programmable gate array to include the indicated version of the variant analysis model for performing genomic analysis. In some cases, the act 706 involves automatically installing the indicated version of the variant analysis model by utilizing a variant analysis model manager to determine available versions of the variant analysis model and to initiate installation of the indicated version from the available versions.

In one or more embodiments, the series of acts 700 includes an act of determining that the indicated version of the variant analysis model is different from the previously installed version of the variant analysis model, wherein only a single version of the variant analysis model can be installed at a time. In these or other embodiments, the act 706 can involve installing the indicated version of the variant analysis model is based on determining that the indicated version is different from the previously installed version. In one or more cases, the act 706 involves replacing the previously installed version of the variant analysis model with the indicated version of the variant analysis model. In certain cases, the act 706 involves installing the indicated version of the variant analysis model in addition to the previously installed version such that the indicated version and the previously installed version of the variant analysis model are installed on one or more servers of the system.

In one or more embodiments, the act 706 involves utilizing a variant analysis model manager housed on a server shared by the variant analysis model to initiate installation of the indicated version of the variant analysis model. The act 706 can involve determining that the indicated version of the variant analysis model is stored within a remote repository storing multiple versions of the variant analysis model. In some cases, the act 706 can involve providing instructions to a server to install the indicated version of the variant analysis model based on determining that the indicated version is stored within the remote repository.

In some embodiments, the series of acts 700 includes an act of determining that the indicated version of the variant analysis model installed on a genomic analysis device is different from the previously installed version of the variant analysis model, wherein the genomic analysis device can be configured to execute a single version of the variant analysis model at a time. The act 706 can further involve installing the indicated version of the variant analysis model on the genomic analysis device based on determining that the indicated version is different from the previously installed version. The series of acts 700 can also include an act of selecting a genomic analysis device as a location for installing the indicated version of the variant analysis model by utilizing a mutating webhook controller to identify a resource label within the application specification that specifies the genomic analysis device.

As further illustrated in FIG. 7, the series of acts 700 includes an act 708 of executing the genomic analysis application utilizing the indicated version of the variant analysis model. In particular, the act 708 can include executing the genomic analysis application to analyze the nucleotide base calls utilizing the indicated version of the variant analysis model. In some embodiments, the act 708 involves determining computational availability of a genomic analysis device housing the variant analysis model for executing the genomic analysis application and scheduling execution of the genomic analysis application by the genomic analysis device based on the computational availability of the genomic analysis device. In certain cases, the series of acts 700 includes an act of receiving sequencing data comprising the nucleotide base calls from a sequencing device. The act 708 can further involve executing the genomic analysis application to analyze the nucleotide base calls utilizing a field programmable gate array configured to execute the indicated version of the variant analysis model.

In one or more embodiments, the series of acts 700 includes an act of identifying multiple workflow pods, wherein two or more of the multiple workflow pods specify different versions of the variant analysis model to perform their respective functions. In these or other embodiments, the series of acts 700 includes an act of iteratively installing the different versions of the variant analysis model for executing each of the multiple workflow pods in series. The series of acts 700 can include an act of utilizing a mutating webhook controller to identify the indicated version of the variant analysis model and to modify an application specification for the genomic analysis application to include instructions for initializing installation of the indicated version of the variant analysis model. Modifying the application specification can include adding an initialization workflow container to the application specification that communicates with a variant analysis model manager to install the indicated version of the variant analysis model.

In certain embodiments, the series of acts 700 includes an act of identifying multiple additional genomic analysis applications for analyzing the nucleotide base calls determined for the sample nucleotide sequence, wherein each of the multiple additional genomic analysis applications specifies different versions of the variant analysis model. The series of acts 700 can also include an act of sequentially executing each of the multiple additional genomic analysis applications by iteratively: installing a version of the variant analysis model for a current genomic analysis application of the multiple additional genomic analysis applications to replace a version from a previous genomic analysis application of the multiple additional genomic analysis applications and executing the current genomic analysis application utilizing the version of the variant analysis model for the current genomic analysis application.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0107082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in U.S. Ser. No. 13/273,666, which is incorporated herein by reference.

The sequencing system described above sequences nucleic acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the model switching system 108 can include software, hardware, or both. For example, the components of the model switching system 108 can include one or more instructions stored on a computer-readable storage medium and executable by processors of one or more computing devices (e.g., the client device 110, the local server device(s) 102, and/or the server device(s) 120). When executed by the one or more processors, the computer-executable instructions of the model switching system 108 can cause the computing devices to perform the bubble detection methods described herein. Alternatively, the components of the model switching system 108 can comprise hardware, such as special purpose processing devices to perform a certain function or group of functions. Additionally, or alternatively, the components of the model switching system 108 can include a combination of computer-executable instructions and hardware.

Furthermore, the components of the model switching system 108 performing the functions described herein with respect to the model switching system 108 may, for example, be implemented as part of a stand-alone application, as a module of an application, as a plug-in for applications, as a library function or functions that may be called by other applications, and/or as a cloud-computing model. Thus, components of the model switching system 108 may be implemented as part of a stand-alone application on a personal computing device or a mobile device. Additionally, or alternatively, the components of the model switching system 108 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 8 illustrates a block diagram of a computing device 800 that may be configured to perform one or more of the processes described above. One will appreciate that one or more computing devices such as the computing device 800 may implement the model switching system 108 and the genomic analysis platform 104. As shown by FIG. 8, the computing device 800 can comprise a processor 802, a memory 804, a storage device 806, an I/O interface 808, and a communication interface 810, which may be communicatively coupled by way of a communication infrastructure 812. In certain embodiments, the computing device 800 can include fewer or more components than those shown in FIG. 8. The following paragraphs describe components of the computing device 800 shown in FIG. 8 in additional detail.

In one or more embodiments, the processor 802 includes hardware for executing instructions, such as those making up a computer program. As an example, and not by way of limitation, to execute instructions for dynamically modifying workflows, the processor 802 may retrieve (or fetch) the instructions from an internal register, an internal cache, the memory 804, or the storage device 806 and decode and execute them. The memory 804 may be a volatile or non-volatile memory used for storing data, metadata, and programs for execution by the processor(s). The storage device 806 includes storage, such as a hard disk, flash disk drive, or other digital storage device, for storing data or instructions for performing the methods described herein.

The I/O interface 808 allows a user to provide input to, receive output from, and otherwise transfer data to and receive data from computing device 800. The I/O interface 808 may include a mouse, a keypad or a keyboard, a touch screen, a camera, an optical scanner, network interface, modem, other known I/O devices or a combination of such I/O interfaces. The I/O interface 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 808 is configured to provide graphical data to a display for presentation to a user. The graphical data may be representative of one or more graphical user interfaces and/or any other graphical content as may serve a particular implementation.

The communication interface 810 can include hardware, software, or both. In any event, the communication interface 810 can provide one or more interfaces for communication (such as, for example, packet-based communication) between the computing device 800 and one or more other computing devices or networks. As an example, and not by way of limitation, the communication interface 810 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 810 may facilitate communications with various types of wired or wireless networks. The communication interface 810 may also facilitate communications using various communication protocols. The communication infrastructure 812 may also include hardware, software, or both that couples components of the computing device 800 to each other. For example, the communication interface 810 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

As used herein, the term “object” includes all things that are suitable for imaging, viewing, analyzing, inspecting, or profiling with the optical systems described herein. By way of example only, objects may include semiconductor wafers or chips, recordable media, samples, flow cells, microparticles, slides, or microarrays. Objects generally include one or more surfaces and/or one or more interfaces that a user may desire to image, view, analyze, inspect, and/or determine a profile thereof. The objects may have surfaces or interfaces with relief features such as wells, pits, ridges, bumps, beads or the like.

As indicated above in the description of a “sample,” a sample may be imaged or scanned for subsequent analysis. In particular embodiments, a sample may include biological or chemical substances of interests and, optionally, an optical substrate that supports the biological or chemical substances. As such, a sample may or may not include an optical substrate. As used herein, the term “biological or chemical substances” is not intended to be limiting, but may include a variety of biological or chemical substances that are suitable for being imaged or examined with the optical systems described herein. For example, biological or chemical substances include biomolecules, such as nucleosides, nucleic acids, polynucleotides, oligonucleotides, proteins, enzymes, polypeptides, antibodies, antigens, ligands, receptors, polysaccharide, carbohydrate, polyphosphates, nanopores, organelles, lipid layers, cells, tissues, organisms, and biologically active chemical compound(s) such as analogs or mimetics of the aforementioned species.

The biological or chemical substances may be supported by an optical substrate. As used herein, the term “optical substrate” is not intended to be limiting, but may include various materials that support the biological or chemical substances and permit the biological or chemical substances to be at least one of viewed, imaged, and examined. For example, the optical substrate may comprise a transparent material that reflects a portion of incident light and refracts a portion of the incident light. Alternatively, the optical substrate may be, for example, a mirror that reflects the incident light entirely such that no light is transmitted through the optical substrate. Typically, the optical substrate has a flat surface. However, the optical substrate can have a surface with relief features such as wells, pits, ridges, bumps, beads or the like.

In an exemplary embodiment, the optical substrate is a flow cell having flow channels where nucleic acids are sequenced. However, in alternative embodiments, the optical substrate may include one or more slides, planar chips (such as those used in microarrays), or microparticles. In such cases where the optical substrate includes a plurality of microparticles that support the biological or chemical substances, the microparticles may be held by another optical substrate, such as a slide or grooved plate. In particular embodiments, the optical substrate includes diffraction grating based encoded optical identification elements similar to or the same as those described in pending U.S. patent application Ser. No. 10/661,234, entitled Diffraction Grating Based Optical Identification Element, filed Sep. 12, 2003, which is incorporated herein by reference in its entirety, discussed more hereinafter. A bead cell or plate for holding the optical identification elements may be similar to or the same as that described in pending U.S. patent application Ser. No. 10/661,836, entitled “Method and Apparatus for Aligning Microbeads in Order to Interrogate the Same”, filed Sep. 12, 2003, and U.S. Pat. No. 7,164,533, entitled “Hybrid Random Bead/Chip Based Microarray”, issued Jan. 16, 2007, as well as U.S. patent application Ser. No. 60/609,583, entitled “Improved Method and Apparatus for Aligning Microbeads in Order to Interrogate the Same”, filed Sep. 13, 2004, Ser. No. 60/1010,910, entitled “Method and Apparatus for Aligning Microbeads in Order to Interrogate the Same”, filed Sep. 17, 2004, each of which is incorporated herein by reference in its entirety.

As used herein, the term “optical components” or “focus components” includes various elements that affect the transmission of light. Optical components may be, for example, reflectors, dichroics, beam splitters, collimators, lenses, filters, wedges, prisms, mirrors, and the like.

By way of example, optical systems described herein may be constructed to include various components and assemblies as described in PCT application PCT/US07/07991, entitled “System and Devices for Sequence by Synthesis Analysis”, filed Mar. 30, 2007 and/or to include various components and assemblies as described in PCT application PCT/US2008/077850, entitled “Fluorescence Excitation and Detection System and Method”, filed Sep. 26, 2008, both of which the complete subject matter are incorporated herein by reference in their entirety. In particular embodiments, optical systems can include various components and assemblies as described in U.S. Pat. No. 7,329,860, of which the complete subject matter is incorporated herein by reference in its entirety. Optical systems can also include various components and assemblies as described in U.S. patent application Ser. No. 12/638,770, filed on Dec. 15, 2009, of which the complete subject matter is incorporated herein by reference in its entirety.

In particular embodiments, methods, and optical systems described herein may be used for sequencing nucleic acids. For example, sequencing-by-synthesis (SBS) protocols are particularly applicable. In SBS, a plurality of fluorescently labeled modified nucleotides are used to sequence dense clusters of amplified DNA (possibly millions of clusters) present on the surface of an optical substrate (e.g., a surface that at least partially defines a channel in a flow cell). The flow cells may contain nucleic acid samples for sequencing where the flow cells are placed within the appropriate flow cell holders. The samples for sequencing can take the form of single nucleic acid molecules that are separated from each other so as to be individually resolvable, amplified populations of a nucleic acid molecules in the form of clusters or other features, or beads that are attached to one or more molecules of nucleic acid. The nucleic acids can be prepared such that they comprise an oligonucleotide primer adjacent to an unknown target sequence. To initiate the first SBS sequencing cycle, one or more differently labeled nucleotides, and DNA polymerase, etc., can be flowed into/through the flow cell by a fluid flow subsystem (not shown). Either a single type of nucleotide can be added at a time, or the nucleotides used in the sequencing procedure can be specially designed to possess a reversible termination property, thus allowing each cycle of the sequencing reaction to occur simultaneously in the presence of several types of labeled nucleotides (e.g., A, C, T, G). The nucleotides can include detectable label moieties such as fluorophores. Where the four nucleotides are mixed together, the polymerase is able to select the correct base to incorporate and each sequence is extended by a single base. One or more lasers may excite the nucleic acids and induce fluorescence. The fluorescence emitted from the nucleic acids is based upon the fluorophores of the incorporated base, and different fluorophores may emit different wavelengths of emission light. Exemplary sequencing methods are described, for example, in Bentley et al., Nature 456:53-59 (2008), WO 04/018497; U.S. Pat. No. 7,057,026; WO 91/06678; WO 07/123,744; U.S. Pat. Nos. 7,329,492; 7,211,414; 7,315,019; 7,405,281, and US 2008/0108082, each of which is incorporated herein by reference.

Other sequencing techniques that are applicable for use of the methods and systems set forth herein are pyrosequencing, nanopore sequencing, and sequencing by ligation. Exemplary pyrosequencing techniques and samples that are particularly useful are described in U.S. Pat. Nos. 6,210,891; 6,258,568; 6,274,320 and Ronaghi, Genome Research 11:3-11 (2001), each of which is incorporated herein by reference. Exemplary nanopore techniques and samples that are also useful are described in Deamer et al., Acc. Chem. Res. 35:817-825 (2002); Li et al., Nat. Mater. 2:611-615 (2003); Soni et al., Clin Chem. 53:1996-2001 (2007) Healy et al., Nanomed. 2:459-481 (2007) and Cockroft et al., J. am. Chem. Soc. 130:818-820; and U.S. Pat. No. 7,001,792, each of which is incorporated herein by reference. Any of a variety of samples can be used in these systems such as substrates having beads generated by emulsion PCR, substrates having zero-mode waveguides, substrates having biological nanopores in lipid bilayers, solid-state substrates having synthetic nanopores, and others known in the art. Such samples are described in the context of various sequencing techniques in the references cited above and further in US 2005/0042648; US 2005/0079510; US 2005/0130173; and WO 05/010145, each of which is incorporated herein by reference.

In other embodiments, optical systems described herein may be utilized for detection of samples that include microarrays. A microarray may include a population of different probe molecules that are attached to one or more substrates such that the different probe molecules can be differentiated from each other according to relative location. An array can include different probe molecules, or populations of the probe molecules, that are each located at a different addressable location on a substrate. Alternatively, a microarray can include separate optical substrates, such as beads, each bearing a different probe molecule, or population of the probe molecules, that can be identified according to the locations of the optical substrates on a surface to which the substrates are attached or according to the locations of the substrates in a liquid. Exemplary arrays in which separate substrates are located on a surface include, without limitation, a Sentrix® Array or Sentrix® BeadChip Array available from Illumina®, Inc. (San Diego, Calif.) or others including beads in wells such as those described in U.S. Pat. Nos. 6,266,459, 6,355,431, 6,770,441, and 6,859,570; and PCT Publication No. WO 00/63437, each of which is hereby incorporated by reference. Other arrays having particles on a surface include those set forth in US 2005/0227252; WO 05/033681; and WO 04/024328, each of which is hereby incorporated by reference.

Any of a variety of microarrays known in the art, including, for example, those set forth herein, can be used in embodiments of the invention. A typical microarray contains sites, sometimes referred to as features, each having a population of probes. The population of probes at each site is typically homogenous having a single species of probe, but in some embodiments the populations can each be heterogeneous. Sites or features of an array are typically discrete, being separated with spaces between each other. The size of the probe sites and/or spacing between the sites can vary such that arrays can be high density, medium density or lower density. High density arrays are characterized as having sites separated by less than about 15 μm. Medium density arrays have sites separated by about 15 to 30 μm, while low density arrays have sites separated by greater than 30 μm. An array useful in the invention can have sites that are separated by less than 100 μm, 50 μm, 10 μm, 5 μm, 1 μm, or 0.5 μm. An apparatus or method of an embodiment of the invention can be used to image an array at a resolution sufficient to distinguish sites at the above densities or density ranges.

Further examples of commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies as described, for example, in U.S. Pat. Nos. 5,324,633; 5,744,305; 5,451,683; 5,482,867; 5,491,074; 5,624,711; 5,795,716; 5,831,070; 5,856,101; 5,858,659; 5,874,219; 5,968,740; 5,974,164; 5,981,185; 5,981,956; 6,025,601; 6,033,860; 6,090,555; 6,136,269; 6,022,963; 6,083,697; 6,291,183; 6,309,831; 6,416,949; 6,428,752 and 6,482,591, each of which is hereby incorporated by reference. A spotted microarray can also be used in a method according to an embodiment of the invention. An exemplary spotted microarray is a CodeLink™ Array available from Amersham Biosciences. Another microarray that is useful is one that is manufactured using inkjet printing methods such as SurePrint™ Technology available from Agilent Technologies.

The systems and methods set forth herein can be used to detect the presence of a particular target molecule in a sample contacted with the microarray. This can be determined, for example, based on binding of a labeled target analyte to a particular probe of the microarray or due to a target-dependent modification of a particular probe to incorporate, remove, or alter a label at the probe location. Any one of several assays can be used to identify or characterize targets using a microarray as described, for example, in U.S. Patent Application Publication Nos. 2003/0108867; 2003/0108900; 2003/0170684; 2003/0207295; or 2005/0181394, each of which is hereby incorporated by reference.

Exemplary labels that can be detected in accordance with embodiments of the invention, for example, when present on a microarray include, but are not limited to, a chromophore; luminophore; fluorophore; optically encoded nanoparticles; particles encoded with a diffraction-grating; electrochemiluminescent label such as Ru(bpy)³²⁺; or moiety that can be detected based on an optical characteristic. Fluorophores that may be useful include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dyes, phycoerythin, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066, each of which is hereby incorporated by reference.

In particular embodiments, the optical system can be configured for Time Delay Integration (TDI) for example in line scanning embodiments as described, for example, in U.S. Pat. No. 7,329,860, of which the complete subject matter is incorporated herein by reference in its entirety. By way of example, the optical assembly may have a 0.75 NA lens and a focus accuracy of +/−125 to 500 nm. The resolution can be 50 to 100 nm. The system may be able to obtain 1,000-10,000 measurements/second unfiltered.

Although embodiments are exemplified with regard to detection of samples that includes biological or chemical substances supported by an optical substrate, it will be understood that other samples can be analyzed, examined, or imaged by the embodiments described herein. Other exemplary samples include, but are not limited to, biological specimens such as cells or tissues, electronic chips such as those used in computer processors, or the like. Examples of some of the applications include microscopy, satellite scanners, high-resolution reprographics, fluorescent image acquisition, analyzing and sequencing of nucleic acids, DNA sequencing, sequencing-by-synthesis, imaging of microarrays, imaging of holographically encoded microparticles and the like.

In other embodiments, the optical systems may be configured to inspect an object to determine certain features or structures of the object. For example, the optical systems may be used to inspect a surface of the object, (e.g., semiconductor chip, silicon wafer) to determine whether there are any deviations or defects on the surface.

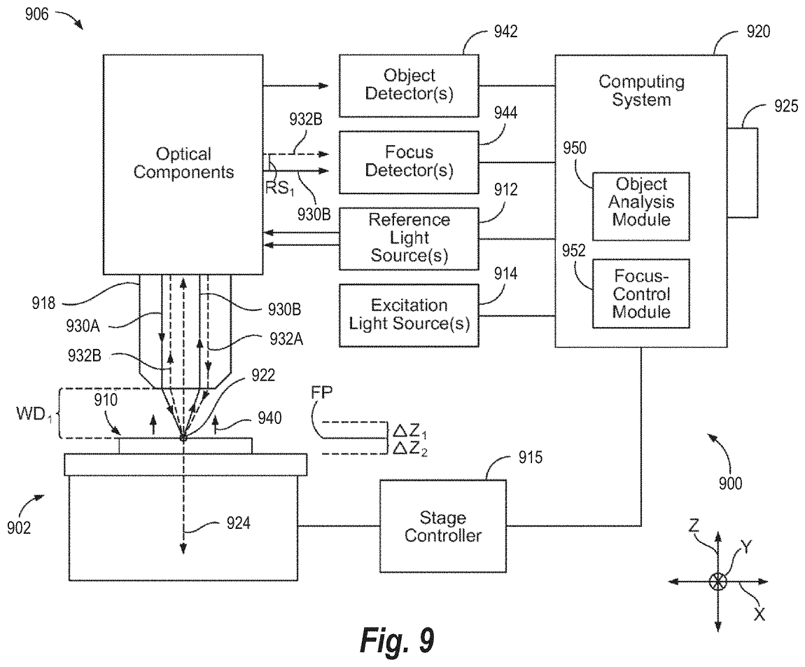

FIG. 9 illustrates a block diagram of an optical system 900 formed in accordance with one embodiment. By way of example only, the optical system 900 may be a sampler imager that images a sample of interest for analysis. In other embodiments, the optical system 900 may be a profilometer that determines a surface profile (e.g., topography) of an object. Furthermore, various other types of optical systems may use the mechanisms and systems described herein. In the illustrated embodiment, the optical system 900 includes an optical assembly 906, an object holder 902 for supporting an object 910 near a focal plane FP of the optical assembly 906, and a stage controller 915 that is configured to move the object holder 902 in a lateral direction (along an X-axis and/or a Y-axis that extend into the page) or in a vertical/elevational direction along a Z-axis. The optical system 900 may also include a system controller or computing system 920 that is operatively coupled to the optical assembly 906, the stage controller 915, and/or the object holder 902.

In particular embodiments, the optical system 900 is a sample imager configured to image samples. Although not shown, a sample imager may include other sub-systems or devices for performing various assay protocols. By way of example only, the sample may include a flow cell having flow channels. The sample imager may include a fluid control system that includes liquid reservoirs that are fluidically coupled to the flow channels through a fluidic network. The sample imager may also include a temperature control system that may have a heater/cooler configured to regulate a temperature of the sample and/or the fluid that flows through the sample. The temperature control system may include sensors that detect a temperature of the fluids.

As shown, the optical assembly 906 is configured to direct input light to an object 910 and receive and direct output light to one or more detectors. The output light may be input light that was at least one of reflected and refracted by the object 910 and/or the output light may be light emitted from the object 910. To direct the input light, the optical assembly 906 may include at least one reference light source 912 and at least one excitation light source 914 that direct light, such as light beams having predetermined wavelengths, through one or more optical components of the optical assembly 906. The optical assembly 906 may include various optical components, including a conjugate lens 918, for directing the input light toward the object 910 and directing the output light toward the detector(s).

In the exemplary embodiment, the reference light source 912 may be used by a distance measuring system or a focus-control system (or focusing mechanism) of the optical system 900 and the excitation light source 914 may be used to excite the biological or chemical substances of the object 910 when the object 910 includes a biological or chemical sample. The excitation light source 914 may be arranged to illuminate a bottom surface of the object 910, such as in TIRF imaging, or may be arranged to illuminate a top surface of the object 910, such as in epi-fluorescent imaging. As shown in FIG. 9, the conjugate lens 918 directs the input light to a focal region 922 lying within the focal plane FP. The lens 918 has an optical axis 924 and is positioned a working distance WD₁ away from the object 910 measured along the optical axis 924. The stage controller 915 may move the object 910 in the Z-direction to adjust the working distance WD₁ so that, for example, a portion of the object 910 is within the focal region 922.

To determine whether the object 910 is in focus (i.e., sufficiently within the focal region 922 or the focal plane FP), the optical assembly 906 is configured to direct at least one pair of light beams to the focal region 922 where the object 910 is approximately located. The object 910 reflects the light beams. More specifically, an exterior surface of the object 910 or an interface within the object 910 reflects the light beams. The reflected light beams then return to and propagate through the lens 918. As shown, each light beam has an optical path that includes a portion that has not yet been reflected by the object 910 and a portion that has been reflected by the object 910. The portions of the optical paths prior to reflection are designated as incident light beams 930A and 932A and are indicated with arrows pointing toward the object 910. The portions of the optical paths that have been reflected by the object 910 are designated as reflected light beams 930B and 932B and are indicated with arrows pointing away from the object 910. For illustrative purposes, the light beams 930A, 930B, 932A, and 932B are shown as having different optical paths within the lens 918 and near the object 910. However, in the exemplary embodiment, the light beams 930A and 932B propagate in opposite directions and are configured to have the same or substantially overlapping optical paths within the lens 918 and near the object 910, and the light beams 930B and 932A propagate in opposite directions and are configured to have the same or substantially overlapping optical paths within the lens 918 and near the object 910.

In the embodiment shown in FIG. 9, light beams 930A, 930B, 932A, and 932B pass through the same lens that is used for imaging. In an alternative embodiment, the light beams used for distance measurement or focus determination can pass through a different lens that is not used for imaging. In this alternative embodiment, the lens 918 is dedicated to passing beams 930A, 930B, 932A, and 932B for distance measurement or focus determination, and a separate lens (not shown) is used for imaging the object 910. Similarly, it will be understood that the systems and methods set forth herein for focus determination and distance measurement can occur using a common objective lens that is shared with the imaging optics or, alternatively, the objective lenses exemplified herein can be dedicated to focus determination or distance measurement.

The reflected light beams 930B and 932B propagate through the lens 918 and may, optionally, be further directed by other optical components of the optical assembly 906. As shown, the reflected light beams 930B and 932B are detected by at least one focus detector 944. In the illustrated embodiment, both reflected light beams 930B and 932B are detected by a single focus detector 944. The reflected light beams may be used to determine relative separation RS₁. For example, the relative separation RS₁ may be determined by the distance separating the beam spots from the impinging reflected light beams 930B and 932B on the focus detector 944 (i.e., a separation distance). The relative separation RS₁ may be used to determine a degree-of-focus of the optical system 900 with respect to the object 910. However, in alternative embodiments, each reflected light beam 930B and 932B may be detected by a separate corresponding focus detector 944 and the relative separation RS₁ may be determined based upon a location of the beam spots on the corresponding focus detectors 944.

If the object 910 is not within a sufficient degree-of-focus, the computing system 920 may operate the stage controller 915 to move the object holder 902 to a desired position. Alternatively or in addition to moving the object holder 902, the optical assembly 906 may be moved in the Z-direction and/or along the XY plane.

For example, the object 910 may be relatively moved a distance ΔZ₁ toward the focal plane FP if the object 910 is located above the focal plane FP (or focal region 922), or the object 910 may be relatively moved a distance ΔZ₂ toward the focal plane FP if the object 910 is located below the focal plane FP (or focal region 922). In some embodiments, the optical system 900 may substitute the lens 918 with another lens 918 or other optical components to move the focal region 922 of the optical assembly 906.

The example set forth above and in FIG. 9 has been presented with respect to a system for controlling focus or for determining degree-of-focus. The system is also useful for determining the working distance WD₁ between the object 910 and the lens 918. In such embodiments, the focus detector 944 can function as a working distance detector and the distance separating the beam spots on the working distance detector can be used to determine the working distance between the object 910 and the lens 918. For ease of description, various embodiments of the systems and methods are exemplified herein with regard to controlling focus or determining degree-of-focus. It will be understood that the systems and methods can also be used to determine the working distance between an object and a lens. Likewise, the systems and methods may also be used to determine a surface profile of an object.

In the exemplary embodiment, during operation, the excitation light source 914 directs input light (not shown) onto the object 910 to excite fluorescently-labeled biological or chemical substances. The labels of the biological or chemical substances provide light signals 940 (also called light emissions) having predetermined wavelength(s). The light signals 940 are received by the lens 918 and then directed by other optical components of the optical assembly 906 to at least one object detector 942. Although the illustrated embodiment only shows one object detector 942, the object detector 942 may comprise multiple detectors. For example, the object detector 942 may include a first detector configured to detect one or more wavelengths of light and a second detector configured to detect one or more different wavelengths of light. The optical assembly 906 may include a lens/filter assembly that directs different light signals along different optical paths toward the corresponding object detectors. Such optical systems are described in further detail by PCT Application No. PCT/US07/07991, entitled “System and Devices for Sequence by Synthesis Analysis”, filed Mar. 30, 2007 and PCT Application No. PCT/US2008/077850, entitled “Fluorescence Excitation and Detection System and Method”, filed Sep. 26, 2008, both of which the complete subject matter are incorporated herein by reference in their entirety.

The object detector 942 communicates object data relating to the detected light signals 940 to the computing system 920. The computing system 920 may then record, process, analyze, and/or communicate the data to other users or computing systems, including remote computing systems through a communication line (e.g., Internet). By way of example, the object data may include imaging data that is processed to generate an image(s) of the object 910. The images may then be analyzed by the computing system and/or a user of the optical system 900. In other embodiments, the object data may not only include light emissions from the biological or chemical substances, but may also include light that is at least one of reflected and refracted by the optical substrate or other components. For example, the light signals 940 may include light that has been reflected by encoded microparticles, such as the holographically encoded optical identification elements described above.

In some embodiments, a single detector may provide both functions as described above with respect to the object and focus detectors 942 and 944. For example, a single detector may detect the reflected light beams 930B and 932B and also the light signals 940.

The optical system 900 may include a user interface 925 that interacts with the user through the computing system 920. For example, the user interface 925 may include a display (not shown) that shows and requests information from a user and a user input device (not shown) to receive user inputs.

The computing system 920 may include, among other things, an object analysis module 950 and a focus-control module 952. The focus-control module 952 is configured to receive focus data obtained by the focus detector 944. The focus data may include signals representative of the beam spots incident upon the focus detector 944. The data may be processed to determine relative separation (e.g., separation distance between the beam spots). A degree-of-focus of the optical system 900 with respect to the object 910 may then be determined based upon the relative separation. In particular embodiments, the working distance WD₁ between the object 910 and lens 918 can be determined. Likewise, the object analysis module 950 may receive object data obtained by the object detectors 942. The object analysis module may process or analyze the object data to generate images of the object.

Furthermore, the computing system 920 may include any processor-based or microprocessor-based system, including systems using microcontrollers, reduced instruction set computers (RISC), application specific integrated circuits (ASICs), field programmable gate array (FPGAs), logic circuits, and any other circuit or processor capable of executing functions described herein. The above examples are exemplary only, and are thus not intended to limit in any way the definition and/or meaning of the term system controller. In the exemplary embodiment, the computing system 920 executes a set of instructions that are stored in one or more storage elements, memories, or modules in order to at least one of obtain and analyze object data. Storage elements may be in the form of information sources or physical memory elements within the optical system 900.

The set of instructions may include various commands that instruct the optical system 900 to perform specific protocols. For example, the set of instructions may include various commands for performing assays and imaging the object 910 or for determining a surface profile of the object 910. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

As described above, the excitation light source 914 generates an excitation light that is directed onto the object 910. The excitation light source 914 may generate one or more laser beams at one or more predetermined excitation wavelengths. The light may be moved in a raster pattern across portions of the object 910, such as groups in columns and rows of the object 910. Alternatively, the excitation light may illuminate one or more entire regions of the object 910 at one time and serially stop through the regions in a “step and shoot” scanning pattern. Line scanning can also be used as described, for example, in U.S. Pat. No. 7,329,860, of which the complete subject matter is incorporated herein by reference in its entirety. The object 910 produces the light signals 940, which may include light emissions generated in response to illumination of a label in the object 910 and/or light that has been reflected or refracted by an optical substrate of the object 910. Alternatively, the light signals 940 may be generated, without illumination, based entirely on emission properties of a material within the object 910 (e.g., a radioactive or chemiluminescent component in the object).

The object and focus detectors 942 and 944 may be, for example photodiodes or cameras. In some embodiments herein, the detectors 942 and 944 may comprise a camera that has a 1 mega pixel CCD-based optical imaging system such as a 1002×1004 CCD camera with 8 gm pixels, which at 20× magnification can optionally image an area of 0.4×0.4 mm per tile using an excitation light that has a laser spot size of 0.5×0.5 mm (e.g., a square spot, or a circle of 0.5 mm diameter, or an elliptical spot, etc.). Cameras can optionally have more or less than 1 million pixels, for example a 4 mega pixel camera can be used. In many embodiments, it is desired that the readout rate of the camera should be as fast as possible, for example the transfer rate can be 10 MHz or higher, for example 20 or 30 MHz. More pixels generally mean that a larger area of surface, and therefore more sequencing reactions or other optically detectable events, can be imaged simultaneously for a single exposure. In particular embodiments, the CCD camera/TIRF lasers may collect about 6400 images to interrogate 1600 tiles (since images are optionally done in 4 different colors per cycle using combinations of filters, dichroics and detectors as described herein). For a 1 Mega pixel CCD, certain images optionally can contain between about 5,000 to 50,000 randomly spaced unique nucleic acid clusters (i.e., images upon the flow cell surface). At an imaging rate of 2 seconds per tile for the four colors, and a density of 25000 clusters per tile, the systems herein can optionally quantify about 45 million features per hour. At a faster imaging rate, and higher cluster density, the imaging rate can be improved. For example, a readout rate of a 20 MHz camera, and a resolved cluster every 20 pixels, the readout can be 1 million clusters per second. A detector can be configured for Time Delay Integration (TDI) for example in line scanning embodiments as described, for example, in U.S. Pat. No. 7,329,860, of which the complete subject matter is incorporated herein by reference in its entirety. Other useful detectors include, but are not limited, to an optical quadrant photodiode detector, such as those having a 2×2 array of individual photodiode active areas fabricated on a single chip, examples of which are available from Pacific Silicon Sensor (Westlake Village, Calif.), or a position sensitive detector such as those having a monolithic PIN photodiode with a uniform resistance in one or two dimensions, examples of which are available from Hamamatsu Photonics, K.K., (Hamamatsu City, Japan).

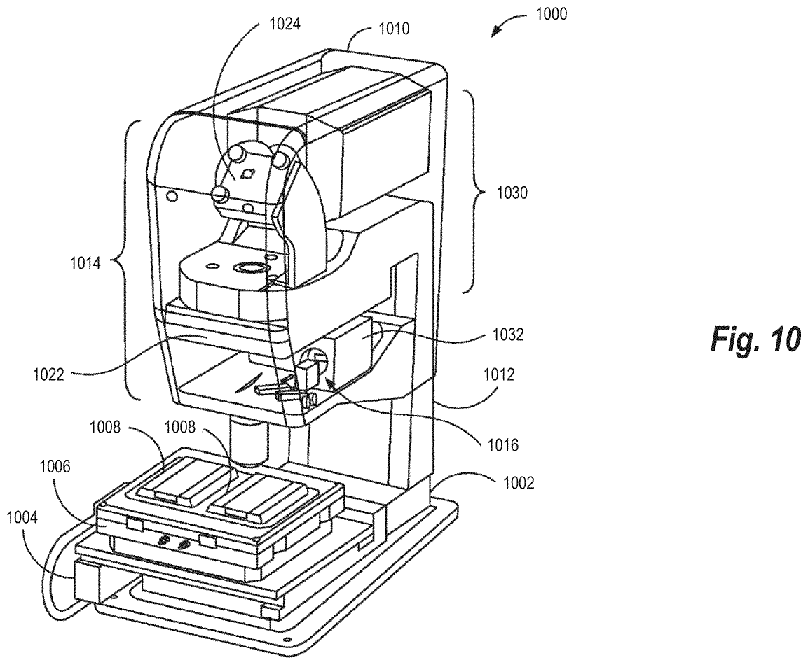

FIG. 10 is a perspective view of a sample imager 1000 formed in accordance with one embodiment. As shown, the sample imager 1000 includes an imager base 1002 that supports a stage 1004 having a sample holder 1006 thereon. The sample holder 1006 is configured to support one or more optical substrates 1008 during an imaging session. The optical substrates 1008 are illustrated as flow cells in FIG. 10. However, other samples may be used.

The sample imager 1000 also includes a housing 1010 (illustrated in phantom) and a strut 1012 that supports the housing 1010. The housing 1010 can enclose at least a portion of an optical assembly 1014 therein. The optical assembly 1014 may include a focus assembly 1016 and a sample-detecting assembly 1030. For example, the focus assembly 1016 may include an auto-focus line scan camera that receives reflected light beams for determining a degree-of-focus of the sampler imager 1000. The sample imager 1000 may also include a filter wheel 1022 and an alignment mirror 1024 that directs light toward a sample detector 1032, which is shown as a K4 camera in FIG. 10.

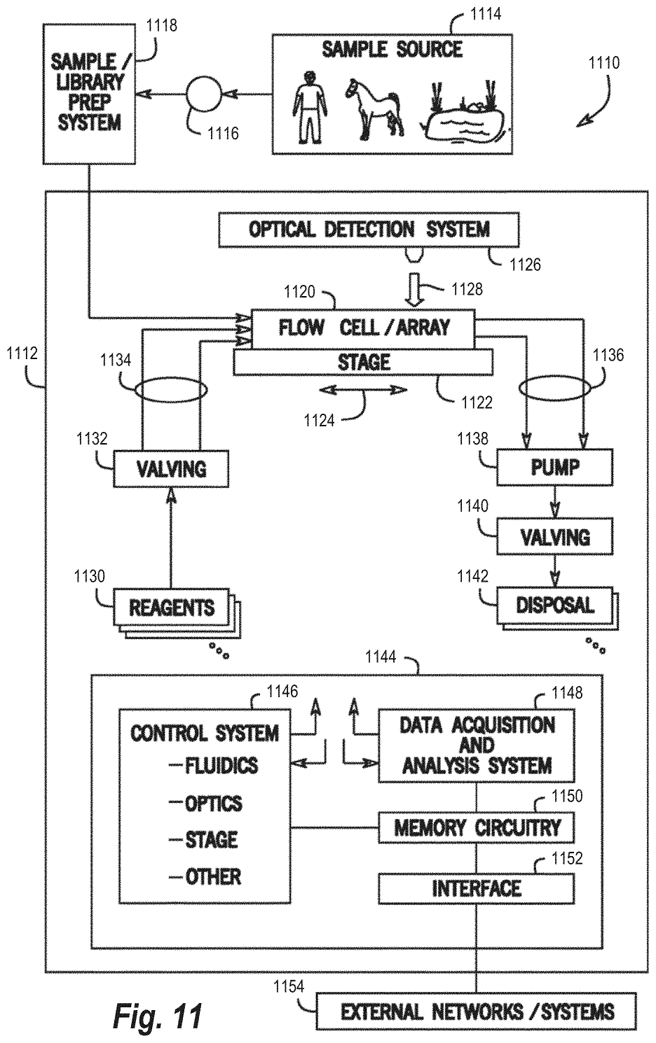

FIG. 11 illustrates an implementation of a sequencing system 1110 configured to process molecular samples that may be sequenced to determine their components, the component ordering, and generally the structure of the sample. The system includes an instrument 1112 that receives and processes a biological sample. A sample source 1114 provides the sample 1116 which in many cases will include a tissue sample. The sample source may include, for example, an individual or subject, such as a human, animal, microorganism, plant, or other donor (including environmental samples), or any other subject that includes organic molecules of interest, the sequence of which is to be determined. Of course, the system may be used with samples other than those taken from organisms, including synthesized molecules. In many cases, the molecules will include DNA, RNA, or other molecules having base pairs the sequence of which may define genes and variants having particular functions of ultimate interest.

The sample 1116 is introduced into a sample/library preparation system 1118. This system may isolate, break, and otherwise prepare the sample for analysis. The resulting library includes the molecules of interest in lengths that facilitate the sequencing operation. The resulting library is then provided to the instrument 1112 where the sequencing operation is performed. In practice, the library, which may sometimes be referred to as a template, is combined with reagents in an automated or semi-automated process, and then introduced to the flow cell prior to sequencing.

In the implementation illustrated in FIG. 11, the instrument includes a flow cell or array 1120 that receives the sample library. The flow cell includes one or more fluidic channels that allow for sequencing chemistry to occur, including attachment of the molecules of the library, and amplification at locations or sites that can be detected during the sequencing operation. For example, the flow cell/array 1120 may include sequencing templates immobilized on one or more surfaces at the locations or sites. A “flow cell” may include a patterned array, such as a microarray, a nanoarray, and so forth. In practice, the locations or sites may be disposed in a regular, repeating pattern, a complex non-repeating pattern, or in a random arrangement on one or more surfaces of a support. To enable the sequencing chemistry to occur, the flow cell also allows for introduction of substances, such as including various reagents, buffers, and other reaction media, that are used for reactions, flushing, and so forth. The substances flow through the flow cell and may contact the molecules of interest at the individual sites.

In the instrument the flow cell 1120 is mounted on a movable stage 1122 that, in this implementation, may be moved in one or more directions as indicated by reference numeral 1124. The flow cell 1120 may, for example, be provided in the form of a removable and replaceable cartridge that may interface with ports on the movable stage 1122 or other components of the system in order to allow reagents and other fluids to be delivered to or from the flow cell 1120. The stage is associated with an optical detection system 1126 that can direct radiation or light 1128 to the flow cell during sequencing. The optical detection system may employ various methods, such as fluorescence microscopy methods, for detection of the analytes disposed at the sites of the flow cell. By way of non-limiting example, the optical detection system 1126 may employ confocal line scanning to produce progressive pixilated image data that can be analyzed to locate individual sites in the flow cell and to determine the type of nucleotide that was most recently attached or bound to each site. Other imaging techniques may also suitably be employed, such as techniques in which one or more points of radiation are scanned along the sample or techniques employing “step and shoot” imaging approaches. The optical detection system 1126 and the stage 1122 may cooperate to maintain the flow cell and detection system in a static relationship while obtaining an area image, or, as noted, the flow cell may be scanned in any suitable mode (e.g., point scanning, line scanning, “step-and-shoot” scanning).

While many different technologies may be used for imaging, or more generally for detecting the molecules at the sites, presently contemplated implementations may make use of confocal optical imaging at wavelengths that cause excitation of fluorescent tags. The tags, excited by virtue of their absorption spectrum, return fluorescent signals by virtue of their emission spectrum. The optical detection system 1126 is configured to capture such signals, to process pixelated image data at a resolution that allows for analysis of the signal-emitting sites, and to process and store the resulting image data (or data derived from it).

In a sequencing operation, cyclic operations or processes are implemented in an automated or semi-automated fashion in which reactions are promoted, such as with single nucleotides or with oligonucleotides, followed by flushing, imaging and de-blocking in preparation for a subsequent cycle. The sample library, prepared for sequencing and immobilized on the flow cell, may undergo a number of such cycles before all useful information is extracted from the library. The optical detection system 1126 may generate image data from scans of the flow cell (and its sites) during each cycle of the sequencing operation by use of electronic detection circuits (e.g., cameras or imaging electronic circuits or chips). The resulting image data may then be analyzed to locate individual sites in the image data, and to analyze and characterize the molecules present at the sites, such as by reference to a specific color or wavelength of light (a characteristic emission spectrum of a particular fluorescent tag) that was detected at a specific location, as indicated by a group or cluster of pixels in the image data at the location. In a DNA or RNA sequencing application, for example, the four common nucleotides may be represented by distinguishable fluorescence emission spectra (wavelengths or wavelength ranges of light). Each emission spectrum, then, may be assigned a value corresponding to that nucleotide. Based upon this analysis, and tracking the cyclical values determined for each site, individual nucleotides and their orders may be determined for each site. These sequences may then be further processed to assemble longer segments including genes, chromosomes, and so forth. As used in this disclosure the terms “automated” and “semi-automated” mean that the operations are performed by system programming or configuration with little or no human interaction once the operations are initiated, or once processes including the operations are initiated.

In the illustrated implementation, reagents 1130 are drawn or aspirated into the flow cell through valving 1132. The valving may access the reagents from recipients or vessels in which they are stored, such as through pipettes or sippers (not shown in FIG. 11). The valving 1132 may allow for selection of the reagents based upon a prescribed sequence of operations performed. The valving may further receive commands for directing the reagents through flow paths 1134 into the flow cell 1120. Exit or effluent flow paths 1136 direct the used reagents from the flow cell. In the illustrated implementation, a pump 1138 serves to move the reagents through the system. The pump may also serve other useful functions, such as measuring reagents or other fluids through the system, aspirating air or other fluids, and so forth. Additional valving 1140 downstream of pump 1138 allows for appropriately directing the used reagent to disposal vessels or recipients 1142.

The instrument further includes a range of circuitry that aids in commanding the operation of the various system components, monitoring their operation by feedback from sensors, collecting image data, and at least partially processing the image data. In the implementation illustrated in FIG. 11, a control/supervisory system 1144 includes a control system 1146 and a data acquisition and analysis system 1148. Both systems will include one or more processors (e.g., digital processing circuits, such as microprocessors, multi-core processors, FPGA's, or any other suitable processing circuitry) and associated memory circuitry 1150 (e.g., solid state memory devices, dynamic memory devices, on and/or off-board memory devices, and so forth) that may store machine-executable instructions for controlling, for example, one or more computers, processors, or other similar logical devices to provide certain functionality. Application-specific or general purpose computers may at least partially make up the control system and the data acquisition and analysis system. The control system may include, for example, circuitry configured (e.g., programmed) to process commands for fluidics, optics, stage control, and any other useful functions of the instrument. The data acquisition and analysis system 1148 interfaces with the optical detection system to command movement of the optical detection system or the stage, or both, the emission of light for cyclic detection, receiving and processing of returned signals, and so forth. The instrument may also include various interfaces as indicated at reference 1152, such as an operator interface that permits control and monitoring of the instrument, transfer of samples, launching of automated or semi-automated sequencing operations, generation of reports, and so forth. Finally, in the implementation of FIG. 11, external networks or systems 1154 may be coupled to and cooperate with the instrument, for example, for analysis, control, monitoring, servicing, and other operations.

It may be noted that while a single flow cell and fluidics path, and a single optical detection system 1126 are illustrated in FIG. 11, in some instruments more than one flow cell and fluidics path may be accommodated. For example, in a presently contemplated implementation, two such arrangements are provided to enhance sequencing and throughput. In practice, any number of flow cells and paths may be provided. These may make use of the same or different reagent receptacles, disposal receptacles, control systems, image analysis systems, and so forth. Where provided, the multiple fluidics systems may be individually controlled or controlled in a coordinated fashion.

We claim:

1. A system comprising:
at least one processor; and
a non-transitory computer readable medium comprising instructions that, when executed by the at least one processor, cause the system to:
identify a genomic analysis application for analyzing nucleotide base calls determined for a sample nucleotide sequence;
determine an indicated version of a variant analysis model for executing the genomic analysis application indicated by an application specification defining one or more parameters for the genomic analysis application;
based on determining the indicated version of the variant analysis model, install the indicated version of the variant analysis model for execution instead of a previously installed version of the variant analysis model; and
execute the genomic analysis application to analyze the nucleotide base calls utilizing the indicated version of the variant analysis model.

2. The system of claim 1, further comprising instructions that, when executed by the at least one processor, cause the system to install the indicated version of the variant analysis model by updating a field programmable gate array to include the indicated version of the variant analysis model for performing genomic analysis.

3. The system of claim 1, further comprising instructions that, when executed by the at least one processor, cause the system to install the indicated version of the variant analysis model automatically by utilizing a variant analysis model manager to determine available versions of the variant analysis model and to initiate installation of the indicated version from the available versions.

4. The system of claim 1, further comprising instructions that, when executed by the at least one processor, cause the system to determine the indicated version of the variant analysis model for executing the genomic analysis application by analyzing the application specification to identify a version label specifying the indicated version of the variant analysis model.

5. The system of claim 1, further comprising instructions that, when executed by the at least one processor, cause the system to:
determine that the indicated version of the variant analysis model is different from the previously installed version of the variant analysis model, wherein only a single version of the variant analysis model can be installed at a time; and
install the indicated version of the variant analysis model based on determining that the indicated version is different from the previously installed version.

6. The system of claim 1, further comprising instructions that, when executed by the at least one processor, cause the system to replace the previously installed version of the variant analysis model with the indicated version of the variant analysis model.

7. The system of claim 1, further comprising instructions that, when executed by the at least one processor, cause the system to install the indicated version of the variant analysis model in addition to the previously installed version such that the indicated version and the previously installed version of the variant analysis model are installed on one or more servers of the system.

8. A computer-implemented method comprising:
identifying a genomic analysis application for analyzing nucleotide base calls determined for a sample nucleotide sequence;
determining an indicated version of a variant analysis model for executing the genomic analysis application indicated by an application specification defining one or more parameters for the genomic analysis application;
based on determining the indicated version of the variant analysis model, installing the indicated version of the variant analysis model for execution instead of a previously installed version of the variant analysis model; and
executing the genomic analysis application to analyze the nucleotide base calls utilizing the indicated version of the variant analysis model.

9. The computer-implemented method of claim 8, wherein installing the indicated version of the variant analysis model comprises utilizing a variant analysis model manager housed on a server shared by the variant analysis model to initiate installation of the indicated version of the variant analysis model.

10. The computer-implemented method of claim 8, further comprising:
determining computational availability of a genomic analysis device housing the variant analysis model for executing the genomic analysis application; and
scheduling execution of the genomic analysis application by the genomic analysis device based on the computational availability of the genomic analysis device.

11. The computer-implemented method of claim 8, further comprising:
identifying multiple workflow pods, wherein two or more of the multiple workflow pods specify different versions of the variant analysis model to perform their respective functions; and
iteratively installing the different versions of the variant analysis model for executing each of the multiple workflow pods in series.

12. The computer-implemented method of claim 8, further comprising utilizing a mutating webhook controller to identify the indicated version of the variant analysis model and to modify an application specification for the genomic analysis application to include instructions for initializing installation of the indicated version of the variant analysis model.

13. The computer-implemented method of claim 12, wherein modifying the application specification comprises adding an initialization workflow container to the application specification that communicates with a variant analysis model manager to install the indicated version of the variant analysis model.

14. The computer-implemented method of claim 8, wherein determining the indicated version of the variant analysis model for executing the genomic analysis application comprises utilizing a variant analysis model manager to analyze the application specification to identify a version label specifying the indicated version of the variant analysis model.

15. A non-transitory computer readable medium comprising instructions that, when executed by at least one processor, cause a system to:
identify a genomic analysis application for analyzing nucleotide base calls determined for a sample nucleotide sequence;
determine an indicated version of a variant analysis model for executing the genomic analysis application indicated by an application specification defining one or more parameters for the genomic analysis application;
based on determining the indicated version of the variant analysis model, install the indicated version of the variant analysis model for execution instead of a previously installed version of the variant analysis model; and
execute the genomic analysis application to analyze the nucleotide base calls utilizing the indicated version of the variant analysis model.

16. The non-transitory computer readable medium of claim 15, further comprising instructions that, when executed by the at least one processor, cause the system to install the indicated version of the variant analysis model by:
determining that the indicated version of the variant analysis model is stored within a remote repository storing multiple versions of the variant analysis model; and
providing instructions to a server to install the indicated version of the variant analysis model based on determining that the indicated version is stored within the remote repository.

17. The non-transitory computer readable medium of claim 15, further comprising instructions that, when executed by the at least one processor, cause the system to:
identify multiple additional genomic analysis applications for analyzing the nucleotide base calls determined for the sample nucleotide sequence, wherein each of the multiple additional genomic analysis applications specifies different versions of the variant analysis model; and
sequentially execute each of the multiple additional genomic analysis applications by iteratively:
installing a version of the variant analysis model for a current genomic analysis application of the multiple additional genomic analysis applications to replace a version from a previous genomic analysis application of the multiple additional genomic analysis applications; and
executing the current genomic analysis application utilizing the version of the variant analysis model for the current genomic analysis application.

18. The non-transitory computer readable medium of claim 15, further comprising instructions that, when executed by the at least one processor, cause the system to:
receive sequencing data comprising the nucleotide base calls from a sequencing device; and
execute the genomic analysis application to analyze the nucleotide base calls utilizing a field programmable gate array configured to execute the indicated version of the variant analysis model.

19. The non-transitory computer readable medium of claim 15, further comprising instructions that, when executed by the at least one processor, cause the system to:
determine that the indicated version of the variant analysis model installed on a genomic analysis device is different from the previously installed version of the variant analysis model, wherein the genomic analysis device can be configured to execute a single version of the variant analysis model at a time; and
install the indicated version of the variant analysis model on the genomic analysis device based on determining that the indicated version is different from the previously installed version.

20. The non-transitory computer readable medium of claim 15, further comprising instructions that, when executed by the at least one processor, cause the system to select a genomic analysis device as a location for installing the indicated version of the variant analysis model by utilizing a mutating webhook controller to identify a resource label within the application specification that specifies the genomic analysis device.
